(19) United States
(12) Patent Application Publication
(10) Pub. No.: US 2014/0121555 A1
(43) Pub. Date: May 1, 2014

(54) NEUROMONITORING SYSTEMS AND METHODS

(71) Applicants: Justin Scott, Pasco, WA (US); Merrill Birdno, Kennewick, WA (US)

(72) Inventors: Justin Scott, Pasco, WA (US); Merrill Birdno, Kennewick, WA (US)

(21) Appl. No.: 14/070,107

(22) Filed: Nov. 1, 2013

Related U.S. Application Data
(60) Provisional application No. 61/721,482, filed on Nov. 1, 2012.
(60) Provisional application No. 61/796,207, filed on Nov. 1, 2012.

(51) Int. Cl. A61B 5/04; A61B 5/0488; A61B 5/00

(52) U.S. Cl. CPC A61B 5/04001; A61B 5/7278; A61B 5/7445; A61B 5/0488
USPC 600/546

(57) ABSTRACT

Systems, devices, and methods are described for neuromonitoring. In certain embodiments, a signal includes components of a neuromuscular response detected by a sensor attached to a muscle in communication with a nerve being monitored in connection with a surgical procedure. A first component of the neuromuscular response corresponding to a first time period and a second component of the neuromuscular response corresponding to the first time period are extracted from the output signal, wherein the first and second components are different. A first display device and a second display device are configured to display the first component and the second component, respectively, in a substantially simultaneous fashion during the surgical procedure.

### CROSS-REFERENCE TO RELATED APPLICATIONS

This application claims the benefit of U.S. Provisional Application No. 61/721,482, filed Nov. 1, 2012, and U.S. Provisional Application No. 61/796,207, filed Nov. 1, 2012, each of which is hereby incorporated by reference herein in its entirety.

### BACKGROUND

The risk of injury to a nerve is a concern when performing surgical procedures, such as minimally invasive procedures, within close proximity to the spine or nerves. Surgeons increasingly rely on neuromonitoring techniques to monitor the nerves during such surgeries in order to avoid inadvertently injuring or contacting a nerve. Prior devices have been developed to help surgeons avoid contacting and damaging nerves during these procedures, but improvements are needed for enhancing the accuracy and effectiveness of these devices.

### SUMMARY

Disclosed herein are systems, devices, and methods for neuromonitoring, particularly during surgical procedures.

According to one aspect, the systems, devices, and methods include a surgical monitoring system comprising a processor configured to (a) receive from a nerve detection module an output signal that includes components of a neuromuscular response detected by a sensor attached to a muscle in communication with a nerve being monitored in connection with a surgical procedure, (b) extract from the output signal a first component of the neuromuscular response corresponding to a first time period and a second component of the neuromuscular response corresponding to the first time period, wherein the first and second components are different, and (c) cause a first display device and a second display device to display the first component and the second component, respectively, in a substantially simultaneous fashion during the surgical procedure.

In certain implementations, the first component comprises a waveform corresponding to the neuromuscular response and the second component does not include the waveform. In certain implementations, the second component comprises a numerical value of a current intensity level that elicited the neuromuscular response and the first component does not include the numerical value of the current intensity that elicited the neuromuscular response. In certain implementations, the processor is further configured to receive from a user input module in communication with the processor a user-selected mode and to determine the respective contents of the first component and the second component based the user-selected mode. The user-selected mode may indicate whether the first display is viewed by a monitorist or a surgeon and/or whether the second display is viewed by a monitorist or a surgeon. In certain implementations, the processor is further configured to select the content of the first component for display on the first monitor to include a waveform of the neuromuscular response if the user-selected mode indicates that the first display screen is viewed by a monitorist. In certain implementations, the processor is further configured to select the content of the first component for display on the first monitor to exclude a waveform of the neuromuscular response if the user-selected mode indicates that the first display screen is viewed by a surgeon.

In certain implementations, in response to a user-selected indication that the first display or the second display is to be viewed by a monitorist, the processor causes the respective display to include a visual image of a waveform corresponding to the neuromuscular response. In certain implementations, in response to a user-selected indication that the first display or the second display is to be viewed by a surgeon, the processor causes the respective display to include a visual indicator of one of a distance to the nerve or a current stimulation amplitude that evoked the neuromuscular response.

In certain implementations, the processor comprises a first processing unit configured to receive the output signal and to extract the first component for display by the first display and a second processing unit configured to receive the output signal and to extract the second component for display by the second display. The processor may further comprises a third processing unit configured to cause the nerve detection module to execute a nerve detection algorithm that returns the neuromuscular response, and wherein the neuromuscular response includes at least one of nerve proximity, pedicle integrity, nerve direction, and nerve status.

In certain implementations, the first and second display devices display information using different respective graphical interfaces. The second display device may display at least some, but not all, of the information displayed by the first display device. In certain implementations, the first display has a number of graphic elements and the second display has fewer graphic elements than the first display. In certain implementations, the first display includes text and the second display includes less text than the first display. In certain implementations, the first display has a number of selectable items and the second display has fewer selectable items than the first display.

In certain implementations, the first and second display devices are communicatively coupled using a physical connector. In certain implementations, the first and second display devices are communicatively coupled using a wireless transmission.

In certain implementations, the nerve detection module is coupled to a probe that delivers a current stimulus to provoke a physiological response. In certain implementations, the user of the first display device may be a monitorist and the user of the second display device may be a surgeon. The first display device displays waveform responses for a plurality of muscles, wherein each muscle has a respective sub-window in the first display. In certain implementations, the current stimulus amplitude associated with each waveform response is displayed as a colored watermark in the background of the sub-window for that respective muscle. The first display device may display at least one of the approximate distance to and direction of the nearest nerve. In certain implementations, the second display device indicates the lowest threshold current for any muscle at a given point in time. The lowest threshold may be indicated using a dial, and the dial may include the threshold value in text and a gauge arrow that points to the threshold value on a semi-circular scale. The background color of the dial may change based on predetermined threshold range definitions. In certain implementations, the second display device displays a virtual representation of at least one nerve and a direction from the probe to the nerve. The display of the information in the first and second displays may be customizable by the user.

According to one aspect, a method for evaluating nerve response includes emitting a plurality of stimulus signals from an electrode disposed in the distal region of a probe or surgical tool approaching or placed adjacent to a nerve, with an amplitude of each stimulus signal being increased by a first constant increment from an amplitude of a previous stimulus signal. The signals are emitted until a first stimulus signal from the plurality of stimulus signals evokes a neuromuscular response that exceeds a response threshold in muscle innervated by the nerve. The first stimulus current and a preceding stimulus current define initial upper and lower bounds, respectively, of an initial current range, and the preceding stimulus current does not evoke a neuromuscular response that exceeds the response threshold. The electrode repeatedly emits one or more subsequent stimulus signals each having a different amplitude selected from within the initial current range until a final current range narrower than the initial current range and having a width less than or equal to a predetermined resolution is determined. The final current range has a final upper bound corresponding to a stimulus current that evokes a neuromuscular response that exceeds the response threshold and a final lower bound corresponds to a stimulus current that does not evoke a neuromuscular response that exceeds the response threshold.

In certain implementations, a beginning stimulus current of the one or more subsequent stimulus currents is delivered at a current amplitude that is not a midpoint current of the initial current range. The beginning stimulus current is based on an amplitude of a neuromuscular response to the first stimulus current. The method may further comprise comparing the measured muscle response to the first stimulus current to a plurality of secondary response thresholds, and the beginning stimulus current may be determined from the comparisons.

In certain implementations, repeatedly emitting subsequent stimulus currents includes increasing the stimulus currents starting at the lower bound of the initial current range, and each subsequent stimulus current is increased by a second constant step value smaller than the first constant step value. The subsequent stimulus currents are emitted until a measured muscle response exceeds the response threshold or the subsequent stimulus currents reach the upper bound of the initial current range.

In certain implementations, repeatedly emitting subsequent stimulus currents comprises decreasing the stimulus currents starting at the upper bound of the initial current range, and each subsequent stimulus current is decreased by a second constant step value smaller than the first constant step value. The subsequent stimulus currents are emitted until a measured muscle response does not exceed the response threshold or the subsequent stimulus currents reach the lower bound of the initial current range.

In certain implementations, a second constant step value is about 1 mA. In other implementations, a second constant step value is less than 1 mA. In other implementations, a second constant step value is selected from a range of about 0.25 mA to about 0.75 mA. In other implementations, a constant step value is about 0.25 mA.

In certain implementations, repeatedly emitting the one or more subsequent stimulus currents comprises repeatedly bisecting the initial current range until the final range is determined.

In certain implementations, repeatedly emitting subsequent stimulus signals includes emitting a first one of the subsequent stimulus signals having an amplitude less than the midpoint of the initial current range immediately after the initial range is determined. In other implementations, repeatedly emitting one or more subsequent stimulus signals comprises emitting a first one of the subsequent stimulus signals having an amplitude greater than the midpoint of the initial current range immediately after the initial range is determined.

In certain implementations, the method includes monitoring an impedance sensed by a stimulus probe, and stimulus currents are not delivered until after the sensed impedance is less than a predetermined low impedance threshold.

In certain implementations, the first constant step value is about 4 mA. In other implementations, the first constant step value is selected from the range 4 mA to 10 mA. In other implementations, the first constant step value is selected from a range of about 4 mA to about 8 mA. In other implementations, the first constant step value is less than 4 mA.

In certain implementations, delivering one or more subsequent stimulus currents includes delivering two subsequent stimulus currents. The final current range may have a size of about 1 mA, or may have a size of about 0.5 mA, or may have a size within the range of about 0.25 mA to about 0.75 mA.

In certain implementations, the method includes determining, based on the final upper bound, an estimate of the distance between the nerve and distal region of the probe or surgical tube. The method includes providing a visual indication of the distance. Providing a visual indication of the distance includes providing a color-coded indicator of the distance, and the visual indicator indicates within which of a plurality of predetermined distance ranges the distance falls. A color that corresponds to the predetermined distance range is displayed.

In certain implementations, the method includes displaying the final upper bound as the stimulus signal that evoked the final neuromuscular response.

According to one aspect, a method for monitoring a surgical procedure includes stimulating a nerve with an electrical stimulation current, detecting a muscle response to the electrical stimulation current, and simultaneously displaying information based on the muscle reaction on first and second display devices, each display device being customized to the respective user of the display.

According to one aspect, a method for monitoring a nerve during a surgical procedure includes (a) receiving from a nerve detection module an output signal that includes components of a neuromuscular response detected by a sensor attached to a muscle in communication with a nerve being monitored in connection with a surgical procedure, (b) extracting from the output signal a first component of the neuromuscular response corresponding to a first time period and a second component of the neuromuscular response corresponding to the first time period, wherein the first and second components are different, and (c) causing a first display device and a second display device to display the first component and the second component, respectively, in a substantially simultaneous fashion during the surgical procedure.

According to one aspect, a surgical system includes the surgical monitoring system discussed above adapted to perform the any of the methods discussed above.

Variations and modifications of these embodiments will occur to those of skill in the art after reviewing this disclosure. The foregoing features and aspects may be implemented, in any combination and subcombination (including multiple dependent combinations and subcombinations), with one or more other features described herein. The various features described or illustrated herein, including any components thereof, may be combined or integrated in other systems. Moreover, certain features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 shows an illustrative surgical monitoring system;

FIG. 2 shows an illustrative surgical monitoring system coupled to a surgical instrument and electrode;

FIG. 3 shows illustrative steps for splitting signal components among first and second display devices;

FIGS. 4 and 5 show block diagrams of illustrative surgical monitoring systems;

FIG. 6 shows an illustrative display screen having various mode windows;

FIGS. 7-9 show illustrative display screens having mode windows docked to various regions within the respective display screens;

FIGS. 10 and 11 show various illustrative displays for use with the surgeon view;

FIGS. 12-15 show various illustrative dials displayed during a surgical procedure;

FIG. 16 shows an illustrative display for use with the monitorist view;

FIG. 17 shows an illustrative sub-window indicating the response amplitude threshold;

FIG. 18 shows an illustrative sub-window for artifact rejection;

FIG. 19 shows an illustrative interface for adjusting threshold ranges;

FIG. 20 shows an illustrative process for determining a minimum stimulus current for a nerve;

FIG. 21 shows an illustrative process for determining a minimum stimulus current for a nerve;

FIG. 22 shows an illustrative diagram of an implementation of the process of FIG. 21;

FIGS. 23-25 show illustrative diagrams of resolution processes; and

FIGS. 26 and 27 show illustrative diagrams of processes for determining a stimulus current range.

### DETAILED DESCRIPTION

To provide an overall understanding of the systems, devices, and methods described herein, certain illustrative embodiments will be described. Although the embodiments and features described herein are specifically described for use in connection with spinal surgical procedures, it will be understood that all the system components, connection mechanisms, surgical procedures, and other features outlined below may be combined with one another in any suitable manner and may be adapted and applied to systems to be used in other surgical procedures performed in the proximity of neural structures where nerve avoidance, detection, or mapping is desired, including, but not limited to spine surgeries, brain surgeries, carotid endarterectomy, otolaryngology procedures such as acoustic neuroma resection, parotidectomy, nerve surgery, or any other suitable surgical procedures.

The present disclosure relates to systems, devices, and methods for intraoperative neuromonitoring (IONM) of any of evoked potential (EP), transcranial electrical motor evoked potential (TceMEP), electromyography (EMG), and electroencephalogram (EEG) signals. Intraoperative neuromonitoring reduces the risk of permanent injury to neural structures during surgical procedures. Changes or abnormalities in the recorded signals may indicate that the surgical procedure is affecting the neural structure. The systems, devices, and methods of the present disclosure measure and display the electrical signals generated by any one or more of muscles, the central nervous system, and peripheral nerves and acquire the data necessary to perform intraoperative monitoring of neural pathways to prevent damage to neural structures during surgical procedures. It will be appreciated that the systems, devices, and methods of the present disclosure can be adapted for use in pre- and/or post-operative procedures in addition to or in place of intraoperative procedures.

Electrical nerve assessment can be employed during a lateral approach spinal surgery in which instruments are advanced to the spine in a trans-psoas approach through a user's side. Such an approach may be preferred to gain access to the spine, for example to vertebral pedicles, and to provide advantageous angles for insertion of pedicle screws. Instruments approaching the spine laterally must be advanced with caution, as sensitive nerve roots from the spinal cord exit the spine in lateral directions, and harm or unintentional stimulation of these nerve roots can cause pain or damage. In order to avoid unwanted contact with these nerves, electrical assessment procedures discussed herein may be used to determine the proximity of nerves and warn a surgeon if an instrument is approaching too near to one or more of the nerve roots. By continually applying stimulus currents to the instruments and measuring the responses in muscles innervated by the nerve roots, such processes can guide a surgeon through the lateral muscles and to the spine without unintentionally contacting or damaging the nerves.

These electrical nerve assessment processes may also be used to evaluate and monitor the integrity of a pedicle during tapping, insertion, and final placement of a spinal screw once instruments are advanced to the spine. The pedicles of a vertebra form the medial and lateral boundaries of the canal through the spine that houses the spinal cord, and lateral nerve roots extend outward from the spinal cord near the pedicles. Any screw or other instrument advanced into the pedicle must thus be precisely inserted so as to avoid compromising the walls of the pedicle and exposing the screw or instrument to the sensitive nerve tissue. In order to evaluate the integrity of a pedicle during these sensitive processes, an electrical stimulus and muscle monitoring approach such as the approaches discussed herein may be employed. The bone material that forms the pedicle insulates an interior channel through the pedicle, and instruments placed into the channel, from the sensitive surrounding nerves. Thus, an uncompromised pedicle will prevent surrounding nerves from becoming stimulated by an electrical stimulus applied to the interior channel. However, if the pedicle walls are compromised or nearly compromised during drilling or placement of an instrument, the insulation may be compromised and may result in surrounding nerves being stimulated from an internal stimulus pulse. During or after tapping the pedicle and placing a screw, the electrical assessment procedures discussed herein may be used to apply stimulus to a pedicle or to a screw placed in the pedicle, and responses of muscles innervated by local nerves can be used to identify damaged or compromised pedicles.

Electrical stimulus applied to a patient's tissue can have dangerous complications if the amount of current applied to the tissue is not limited carefully. Application of a high current can cause damage to tissue and nerves within the tissue, and can cause pain to the patient. High currents may cause spasms, seizures, or permanent damage to a patient's muscles or nerves. In order to limit the number of electrical stimuli applied to the tissue, increases between successive stimulations must be sufficiently large that desired response can be found and analyzed without stimulating the patient over and over. This establishes a trade off when increasing consecutive stimuli, as such an increase must be large enough to efficiently reach a desired level but sufficiently small to avoid requiring many stimulations. In an approach where a small increase size is used, the large number of stimulations at small currents may damage the tissue. In an approach where increasing jumps are made between each stimulation, for example where each jump is double the size of a previous jump, larger increases may be used that can cause damage to a patient from one stimulation to the next. Thus, it is desirable to manage the increase between successive jumps, for example by using a moderate constant increase or by using a constant increase at lower currents and larger increases at higher currents.

The present disclosure allows for dual-purpose monitors or displays to be used during a surgical procedure, such as a lateral approach spinal surgery, to help surgeons and others involved in the surgery perform a more accurate procedure. For example, a first monitor may be customized for use by a technician or monitorist and a second monitor may be customized for use by a surgeon. The information displayed on both displays is based on a response to a stimulation current over a given time period and may be customized, for example, to present only the information needed by the respective user (e.g., the surgeon or the monitorist) during the surgery. The information displayed may be automatically selected by the monitoring system and alternatively, or additionally, may be user-configurable. For example, while the monitor used by the monitorist may display the waveforms generated from the application of the current stimulus, such information may be over-inclusive for the surgeon performing the operation, and the monitor used by the surgeon may instead display, in easily-readable text and/or graphic form, only the information needed for the procedure such as the lowest current threshold for any muscle at the particular point in time. The respective displays used by the monitorist and the surgeon are configurable to display any information for the procedure, and in certain embodiments the displays may show the same information depicted in the same manner, although in preferred embodiments the same information, such as the lowest current threshold for muscle, is depicted in different manners as discussed above, for example, as a waveform or a relatively simplified graphical and/or text display. In certain embodiments, an integrated display may combine both the monitorist views and the surgeon views into a single display.

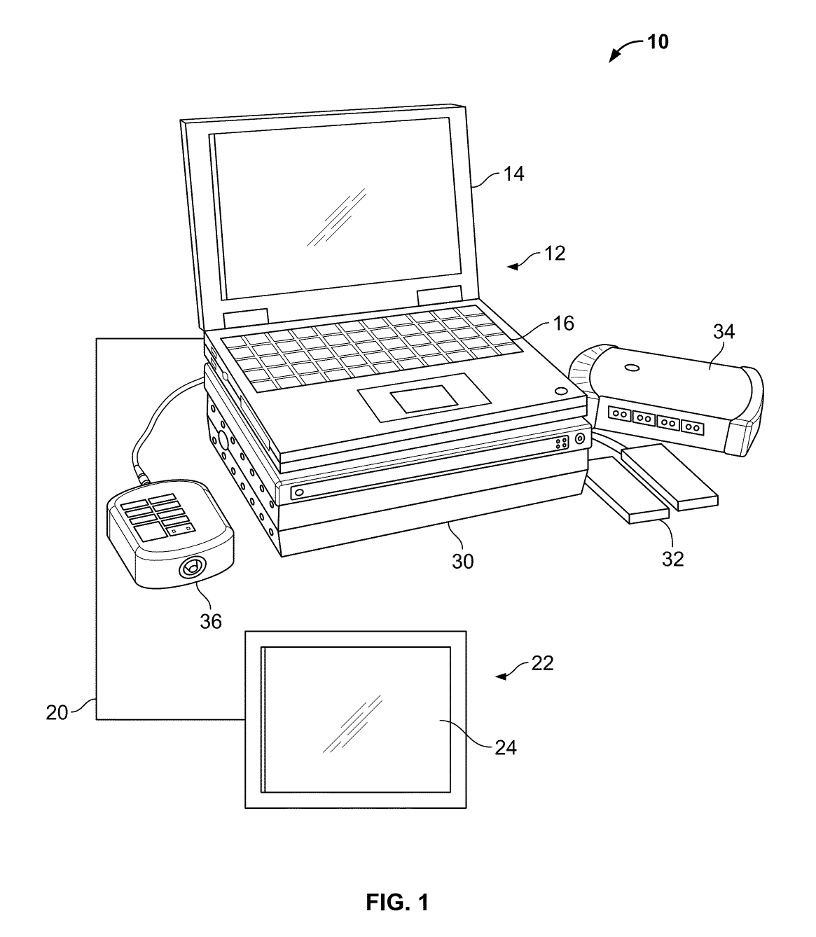

FIG. 1 shows a surgical monitoring system 10 according to certain embodiments. The surgical monitoring system 10 includes a display device 12 having a monitor or display 14 and a user interface 16 for receiving user commands, although in certain embodiments the display 14 includes a touch-screen interface for receiving user inputs. The display device 12 is communicatively coupled to a second display device 22 using a data link 20 that may be a physical connection or a wireless connection. For example, the display devices 12, 22 may be connected to each other by a communication medium, such as a USB port, serial port cable, a coaxial cable, an Ethernet type cable, a telephone line, a radio frequency transceiver or other similar wireless or wired medium or combination of the foregoing. The communication between the display devices 12, 22, and any of the other components in FIG. 1, can follow various known communication protocols, such as TCP/IP, cellular protocols including GSM, Wi-Fi, Wi-Max, or other wireless communications technologies or combination of wired or wireless channels. The second display device 22 includes a monitor or display 24 that may be configured with a touch-screen interface for receiving user inputs or, alternatively or additionally, may be provided with a user interface similar to the user interface 16 shown for the first display device 12. In certain embodiments, the display 24 of the second display device 22 need not include a user input interface.

The display device 12 is coupled to a base unit 30, and one or more of a remote amplifier or 16-channel external amplifier 32, and stimulator 36 (e.g., a Cadwell EX-IX stimulator or stimulator splitter) for measuring and displaying the electrical signals generated by muscles, the central nervous system, and/or the peripheral nerves. Software in the unit allows users to create different procedure setup files for various kinds of surgical cases. During surgery, the user opens the configured setup file to begin a recording session. At the beginning of the surgery, the user opens the configured procedure setup file to begin a recording session. Several modes are run at different intervals during the surgery, collecting data and displaying that data in different formats. The user can run the modes individually or all concurrently. After surgery, the data file may be viewed remotely and the patient data file may be reviewed. Reports can also be created at the end of the surgery and/or when reviewing the data after surgery.

The base unit 30 operates with an electrical stimulator and an evoked potential stimulator (both audio and video). Any suitable electrical stimulator may be used including, for example, the Cadwell ES-IX stimulator, or any of the line of Cadwell stimulator splitters such as the ES5-10, ES5-5 or ES5-5V, ES5-20, ES5-100, and ES-16 stimulator splitters, or any other suitable electrical stimulator. The stimulators have a high current output and a low current output. For example, the ES-IX has a high current output from 0 to 100 mA, 400V maximum, with 0.5 mA resolution below 20 mA and 1 mA resolution above 20 mA. The low current output allows for precise adjustment of low level output currents, and the ES-IX can be configured to various modes of operation, including the following amplitude resolution: 5 mA constant current (0.01 mA resolution), 20 mA constant current (0.1 mA resolution), 5V constant voltage (0.01V resolution), and 20V constant voltage (0.1V resolution). The stimulator splitters allow a single constant-current electrical stimulator output to be multiplexed between multiple stimulation sites. The high current output can range from 0 to 100 mA, 370 VDC maximum, with a current resolution of 0.5 mA (200 steps). The low current output resolution is 0.1 mA or less. As discussed above, any suitable electrical stimulator may be used, and the ranges and outputs can be adjusted according to the algorithms of the present disclosure. Current and voltage feedback is displayed to assist the practitioner in assessing the quality of the stimulator-to-patient connections. Furthermore, in some embodiments, the stimulators may be attached together (e.g., as a daisy chain) to provide a greater number of high current and low current/voltage outputs. The evoked potential stimulators include insert earphones for auditory evoked potentials and/or goggles for visual evoked potentials.

A constant voltage stimulator 34 is optionally coupled to the base unit 30 via an auxiliary port. For example, transcranial stimulators, such as Cadwell Transcranial Stimulators TCS-1/TCS-1000/TCS-4, are constant voltage stimulators for transcranial electrical stimulation for the purpose of recording motor evoked potentials. The devices provide a constant voltage output, and in the case of the TCS-4, one of four channels may be selected as the active output. All of these stimulators include polarity control within the software of the surgical monitoring system. The transcranial stimulators may be coupled to a patient in any suitable manner. For example, the TCS-1/TCS-1000 connects to the patient via a stimulus output pair. The TCS-4 provides the ability to connect four stimulating channels to the patient with one output channel active at any given point in time.

The transcranial stimulators may have various stimulus options including, but not limited to, stimulus polarity, train length, inter stimulus interval (ISI), maximum intensity, repetition rate, double-train stimulation, stimulus pulse width, and channel selection. The stimulus polarity may be normal or reverse, and the devices can produce biphasic stimulation pulses. The transcranial stimulators support multi-pulse train stimulus and single pulse stimulus. The train length setting specifies the number of stimulus pulses delivered in each train, including ranges from 1 to 9, for example. The ISI is the length of time from the start of one stimulus pulse to the start of the next stimulus pulse within a train. An exemplary range is variable from 1.0 to 9.9 milliseconds. The maximum intensity for the TCS-1 is 800V, and the maximum intensity for the TCS-1000 and TCS-4 is 1,000V; variable in 2V increments. The maximum intensity may be set independently for each mode. Furthermore, it will be understood that any suitable voltage maximum and increment may be used. The repetition rate may be set from 0.5 Hz to 1.0 Hz, or any other suitable rate. The double-train stimulation option delivers two trains of stimulation with a user-selected inter-train interval. The stimulus pulse width may be fixed or adjustable. For example, the stimulus pulse width is fixed to 50 microseconds on the TCS-1 and is adjustable to 50 microseconds or 75 microseconds on the TCS-1000/TCS-4. Channel selection allows for various output channels to be selected as the active output. For example, the TCS-4 has an option to select one of the four output channels as the active output.

An electrosurgery detect unit (not shown) is optionally coupled to the base unit 30 via an auxiliary port. The unit is designed to detect the usage of electrical noise generating devices, such as electrosurgery units, and pause data collection while these noisy devices are in use.

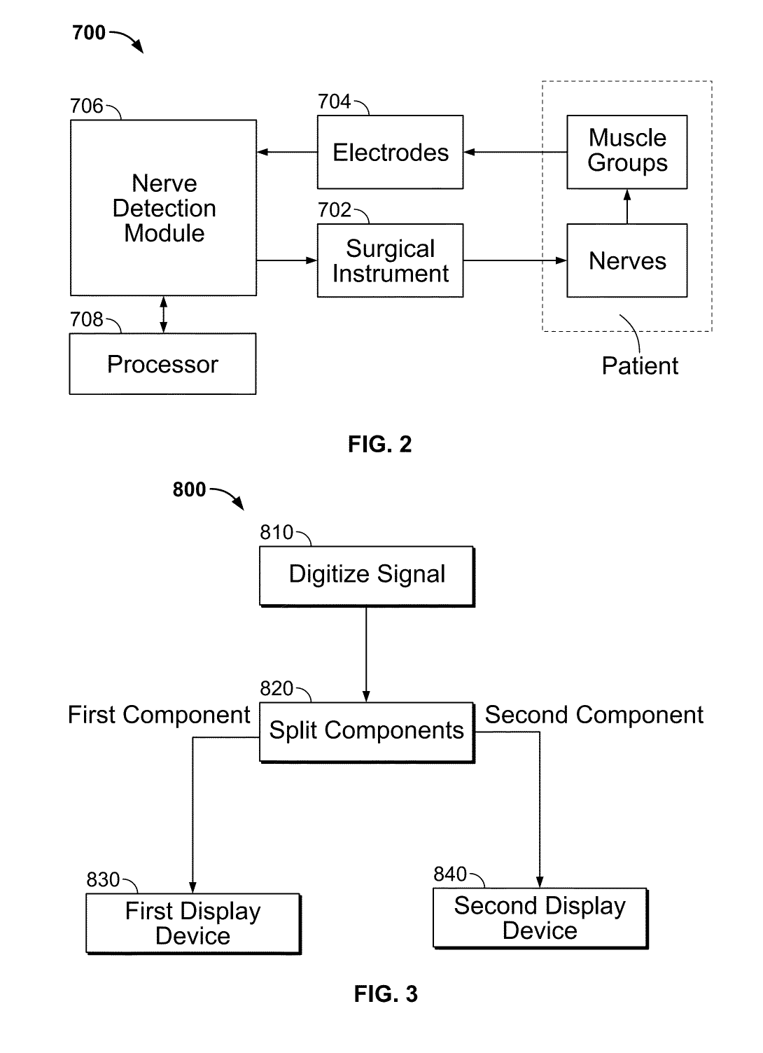

FIG. 2 shows components of a surgical monitoring system 700 according to certain embodiments. The surgical monitoring system 700 is coupled to a surgical instrument 702 for delivering stimulation pulses. The stimulating may be accomplished by applying any of a variety of suitable stimulation signals to an electrode or electrodes on the surgical instrument 702, including voltage and/or current pulses of varying magnitude and/or frequency.

Any suitable surgical instrument may be employed, including, but not limited to, any number of devices or components for creating an operative corridor to a surgical target site (such as K-wires, sequentially dilating cannula systems, distractor systems, and/or retractor systems), devices or components for assessing pedicle integrity (such as a pedicle testing probe), and/or devices or components for retracting or otherwise protecting a nerve root before, during and/or after surgery (such as a nerve root retractor).

Measuring the response of nerves innervated by the stimulation pulses may be performed in any suitable manner, including but not limited to the use of compound muscle action potential (CMAP) monitoring techniques using electrodes 704 coupled to a patient (e.g., measuring the EMG responses of muscle groups associated with a particular nerve). In certain embodiments, measuring the response of nerves is accomplished by monitoring or measuring the EMG responses of the muscles innervated by the stimulated nerves.

The nerve detection module 706 and/or the processor 708 may digitize the signals and split the signal into components for display on first and second display devices according to the steps 800 depicted in FIG. 3. At step 810, signals received by the nerve detection module 706 are digitized. The signals may be received, for example, from electrodes (e.g., electrodes 704) coupled to a patient. At step 820, components of that signal are split into first and second components. The first component is communicated, at step 830, to a first display device, and the second component is communicated, at step 830, to a second display device. Any of the steps 800 of the process can be preformed by the nerve detection module 706 and/or the processor 708. For example, processor 708 may be configured to receive from the nerve detection module 706 an output signal that includes components of a neuromuscular response detected by a sensor (e.g., electrode 704) attached to a muscle in communication with a nerve being monitored in connection with a surgical procedure. A first component of the neuromuscular response corresponding to a first time period is extracted from the output signal, and a second component of the neuromuscular response corresponding to the first time period is extracted from the output signal, where the first and second components are different. The processor 708 may then cause a first display device and a second display device to display the first component and the second component, respectively, in a substantially simultaneous fashion during the surgical procedure.

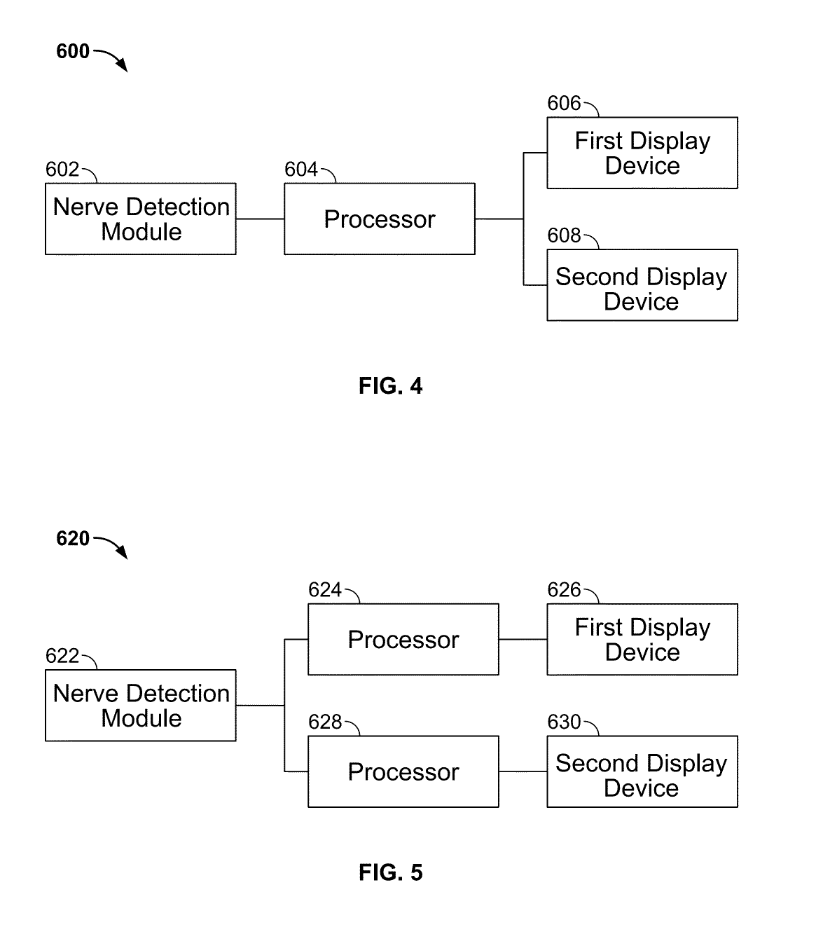

Certain embodiments of systems incorporating the first and second display devices are depicted in FIGS. 4 and 5. As shown in FIG. 4, a nerve detection module 602 is coupled to a processor 604 that delivers signals to the first display device 606 and the second display device 608. The processor 604 may be integrated with one of the first and second display devices 606, 608, or the processor 604 may be integrated with the nerve detection module 602. In certain embodiments, more than one processor may be used. For example, as shown in FIG. 5, a nerve detection module 622 is coupled to a first processor 624 that delivers signals to the first display device 626 and a second processor 628 that delivers signals to the second display device 630. The first and second processors 624, 628 may be integrated with the nerve detection module 622, or the processors 624, 628 may be integrated with the first and second display devices 626, 630, respectively.

The dual-purpose monitors or displays are used during surgical procedures, such as lateral approach spinal surgery, to help surgeons and others involved in the surgery perform a more accurate procedure. Each acquisition mode has a set of specially designed windows from which a user can choose. Each available window type may be identified by its title bar and display data acquired by that mode in a unique way. This allows users (e.g., a surgeon and a monitorist) to view the same data in a variety of ways using the window type most appropriate at any given point in surgery. Several window types can be open at any given time. Furthermore, users can maximize a window so that it fills the entire screen and then restore it to its former size and reposition or minimize that window. The windows can further be moved to different positions and adjusted in size and shape. In certain embodiments, different groups of mode windows can be preconfigured into defined views. This allows users to quickly select a defined view and have the desired groups of mode windows displayed (e.g., upon double-clicking in the screen or other user shortcut using an input device).

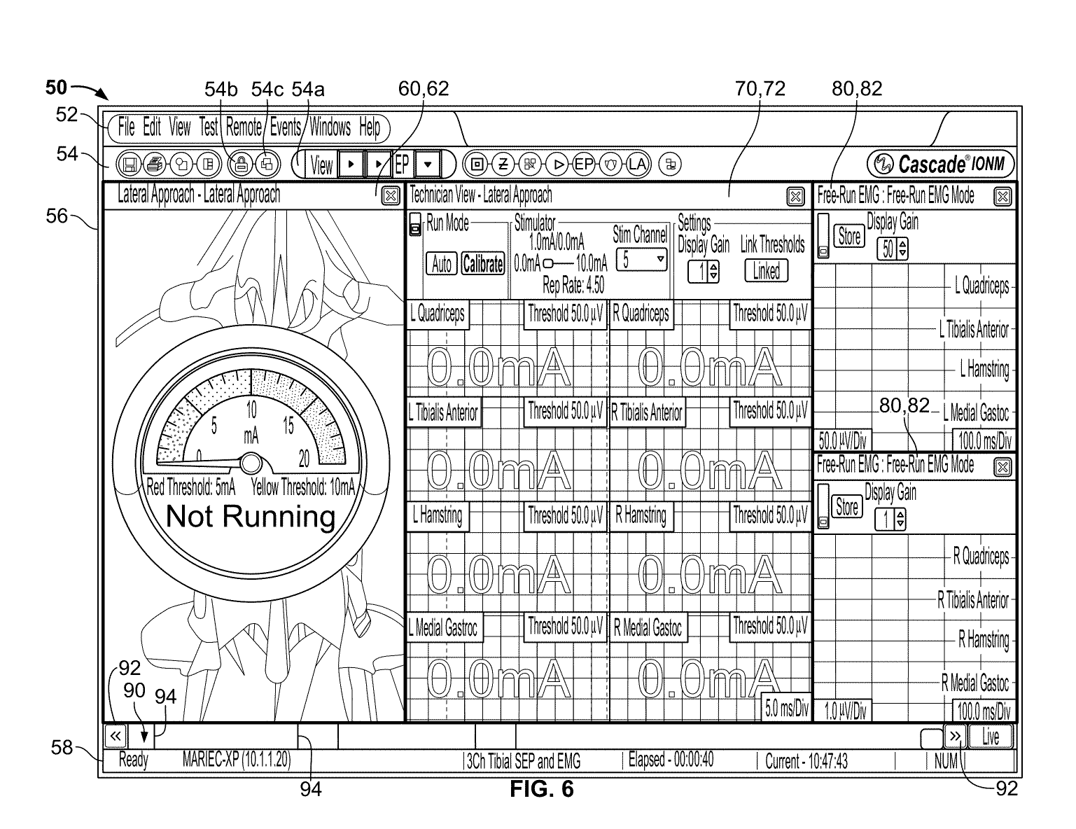

FIG. 6 shows an illustrative display screen 50 according to certain embodiments. The display screen 50 has four basic parts: the menu bar 52, toolbar 54, windows 56, and status bar 58. When performing a procedure or reviewing a procedure, the user is presented with a row of options displayed across the top of the screen as a menu bar 52. The items in this menu bar 52 are the names of menus that, when selected, display a list of commands relating to the name of the menu. For example, windows from any mode can be added to any view by selecting “Edit” and then “View Setup” from the menu bar 52. The buttons on the toolbar 54 are a graphical representation of some of the options available from the menu bar 52. The function of each tool button on the toolbar 54 is described on the status bar 58 when the cursor is over the tool button.

Certain functions of the tool buttons on the toolbar 54 related to viewing the windows 56 will now be described. The view control options 54a allow a user to select views using two command types. The left and right arrows cycle through previously defined views. The view label box indicates the current view (shown as “EP” in the figure). The drop down arrow displays the complete list of views and allows a user to choose the view to display. The lock windows option 54b locks all windows in place. With this option on, the windows cannot be resized or moved. This prevents inadvertent rearranging of windows and increases the amount of space available for waveforms. Selecting the lock windows option 54b again releases the lock. The auto size option 54c resizes surrounding windows as the user resizes or moves one window. This option prevents windows from overlapping and makes it easier to arrange windows.

As discussed above, certain display screens can be integrated to include both the monitorist views and the surgeon views. The display screen 50 includes various windows 56 that display physiological data for a patient according to different modes, including a surgeon window 60, technician or monitorist window 70, and right and left EMG windows 80, and further includes an event timeline 90 along the bottom of the screen. The event timeline 90 includes a right and left arrow 92 for moving between each of the events 94 along the timeline. Each of the windows 60, 70, 80 has a window title bar 62, 72, 82 across the top of the respective window that allows the windows to be docked and undocked from the display screen 50 and placed in any position on a monitor controlled by the monitoring system 10. For example, docking and undocking the surgeon window 60 allows that window to be displayed for the surgeon on a separate monitor such as that provided by the second display device 22 of FIG. 1. The windows may be undocked by grabbing the window title bar using a cursor controlled by a mouse or other input device, including user touch-screen commands, and then moving the window to any position on a monitor controlled by the monitoring system 10.

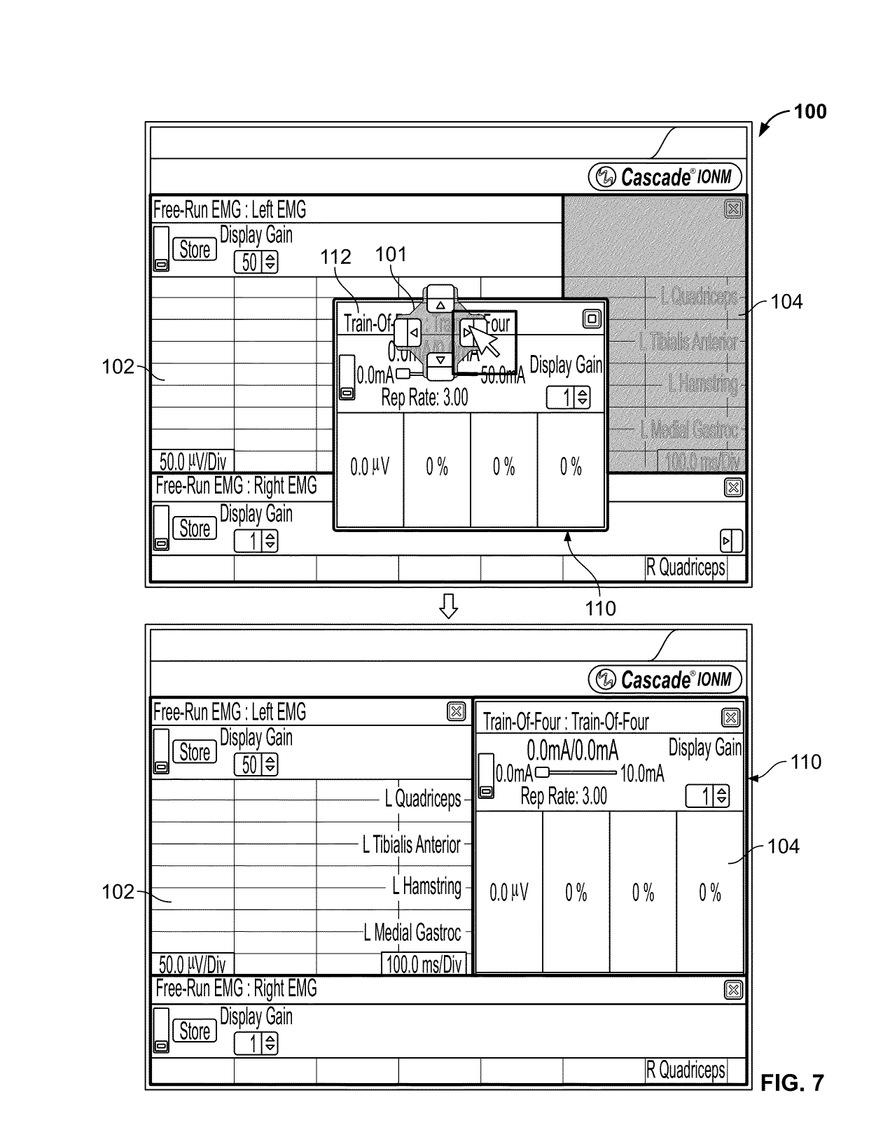

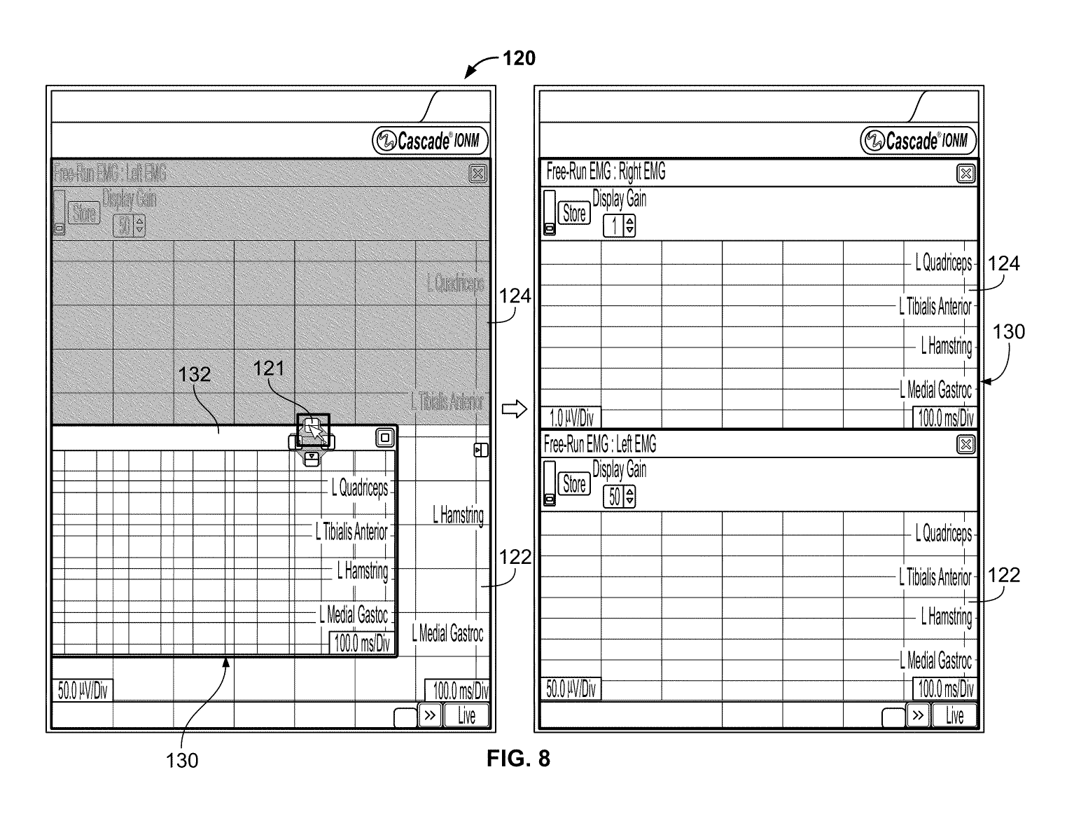

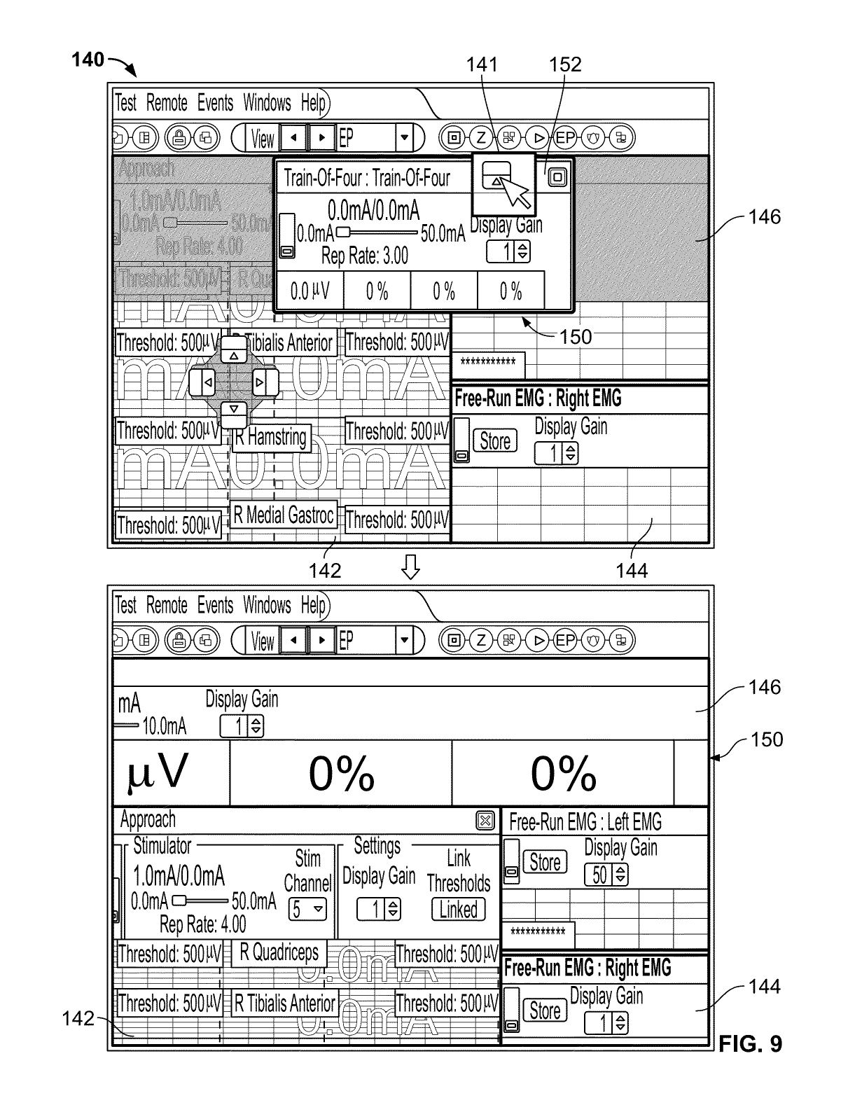

In certain embodiments, to dock an undocked window into a particular region on the display screen, a docking tool is provided that includes a set of arrows that appear when the title bar for that window is selected with the cursor. The potential docking regions for that window will be shadowed in the display screen, and hovering the cursor over different arrows of the docking tool allows the user to see the different docking regions that are available. When a desired docking location is identified, the user releases the title bar and the window becomes docked at the desired docking position. For example, as shown in FIG. 7, a display screen 100 includes a left region 102 and shadowed right region 104 into which the mode window 110 may be docked. When the cursor is positioned over the right arrow of the docking tool 101 and the window title bar 112 is released, the mode window 110 is docked into the right region 104 of the display screen 100. Similarly, as shown in FIG. 8, a display screen 120 includes a bottom region 122 and a shadowed top region 124 into which the mode window 130 may be docked. When the cursor is positioned over the top arrow of the docking tool 121 and the window title bar 132 is released, the mode window 130 is docked into the top region 124 of the display screen 120. As shown in FIG. 9, a mode window can be docked along the top of a display screen 140 having multiple windows. The display screen 140 includes left and right bottom regions 142, 144 and a shadowed top region 146 into which the mode window 150 may be docked. When the cursor is positioned over the top arrow of the docking tool 141 and the window title bar 152 is released, the mode window 150 is docked into the top region 146 of the display screen 140.

The surgical monitoring system allows for simultaneous surgeon and monitorist views of data that is recorded by a nerve detection algorithm. In certain embodiments, this dual-view feature can be implemented by undocking the surgeon window 60 from the integrated view of display screen 50 of FIG. 6 and placing the surgeon window 60 into a second, surgeon-facing, monitor on the second display device 22. It will be understood that any suitable technique may be used to cause the first and second display devices 12, 22 to display the surgeon and monitorist views and that docking and undocking the windows is merely exemplary. In particular, any technique for modifying or otherwise customizing the displays to provide different but simultaneous presentation of a neuromuscular response on different screens may be used. In certain embodiments, the nature of the information displayed in the two (or more) displays depends on a user-selected indication (or automatically determined designation) of the type of user (e.g., monitorist or surgeon).

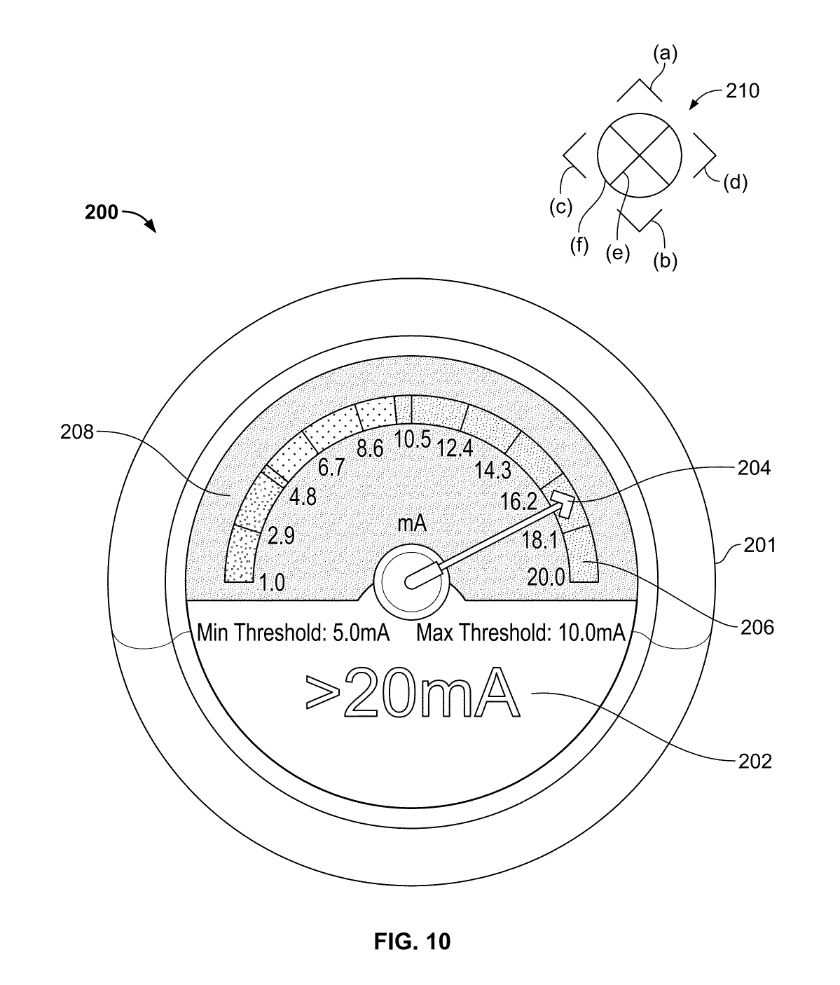

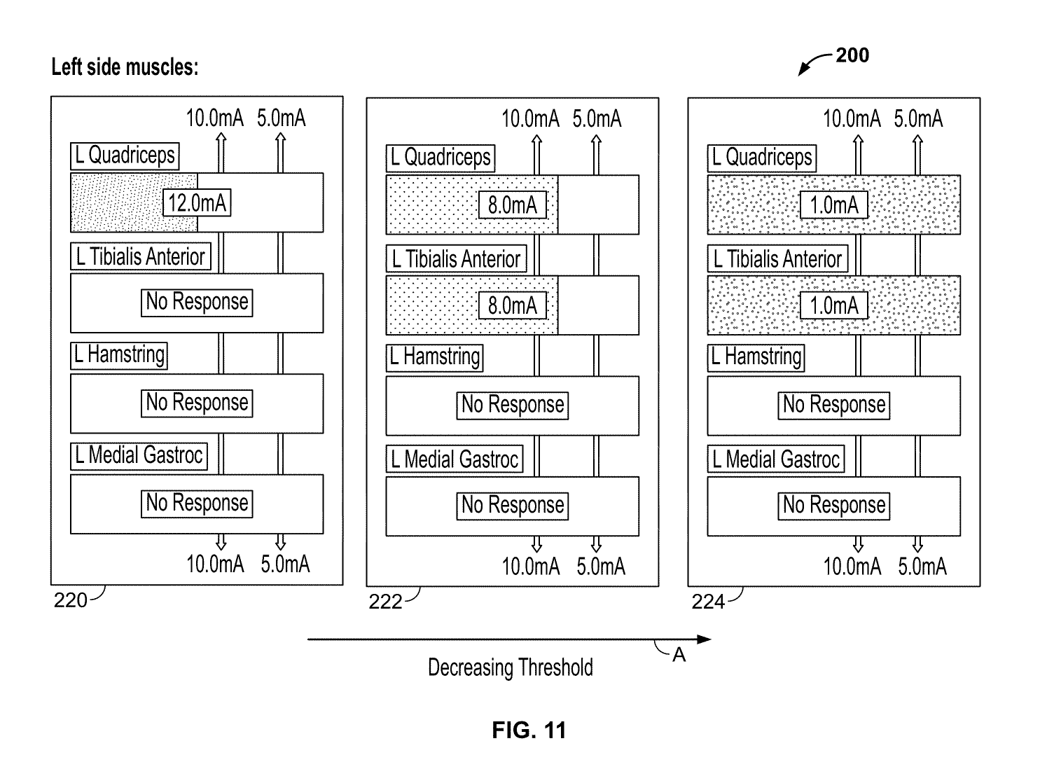

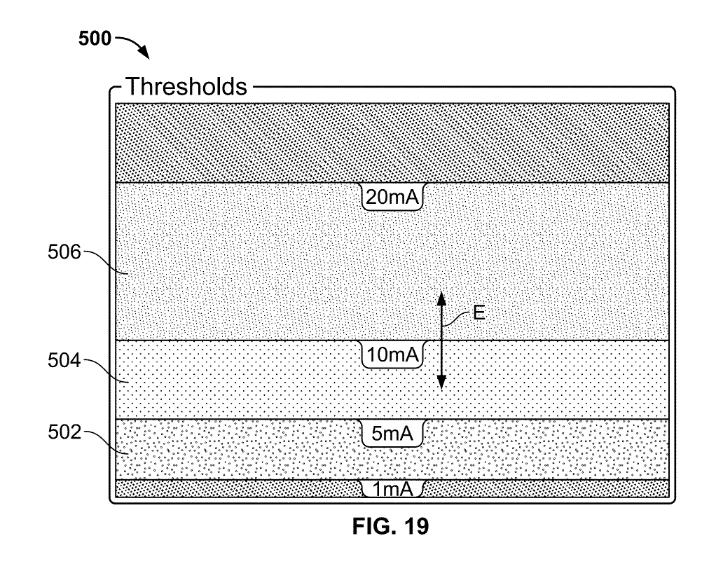

The surgeon view displays information in a relatively simple and easy-to-read manner. For example, as discussed above, the monitorist view may include the waveform responses to the current stimulus while the surgeon view does not; instead including numeric and/or graphical indicators of distance and or current intensity based on the same waveform responses. In certain embodiments, the surgeon view 200 displays information to the surgeon in two respects. First, as shown in FIG. 10, the surgeon view includes a dial 201 that indicates the lowest current threshold value for any sensed muscle at that given point in time. The dial 201 includes the threshold value in large text 202 and a gauge arrow 204 that points to the threshold value on a semi-circular scale 206. The background color 208 of the dial 201 may change according to predetermined range definitions. In certain embodiments, the predetermined range definitions may be configured in a setup screen as shown in FIG. 19. Second, as shown in FIG. 11, the surgeon view 200 displays the individual muscle thresholds via horizontal bar graphs 220, 222, 224 on the left and right sides (or on any other suitable side) of the dial 201. As the threshold for activating a muscle response decreases, the bar increases in size along the direction shown by the decreasing threshold arrow A. In certain embodiments, the bar 220, 222, 224 changes from green to yellow to red (or any other suitable color), as the threshold decreases through the threshold ranges. In certain embodiments, the surgeon dial windows are user-configurable to change the relative size of the respective windows.

In certain embodiments, the dial 201 can be used to indicate to the surgeon the absolute distance to a proximal nerve. Similar to the manner in which the dial 201 indicates the lowest threshold for any sensed muscle, the dial 201 may include the distance value in large text 202 and a gauge arrow 204 that points to the distance value on a semi-circular scale 206. The background color 208 of the dial 201 may change according to predetermined range definitions. In certain embodiments, the predetermined range definitions may be configured in a setup screen.

Furthermore, in certain embodiments, the dial 201 can be used to indicate to the surgeon the direction of a proximal nerve. For example, a directional indicator 210 may be displayed with the dial 201 to indicate the relative direction of the proximal nerve with respect to the travel of the probe in three-dimensions including (a) superior, (b) inferior, (c) medial, (d) lateral, (e) anterior, and (f) posterior directional indicators. Any suitable technique may be used for determining the location of a nerve. Mapping the location of nerves is discussed in detail in U.S. Patent Application Publication No. 2012/0109004, filed Oct. 27, 2010, the disclosure of which is hereby incorporated by reference herein in its entirety.

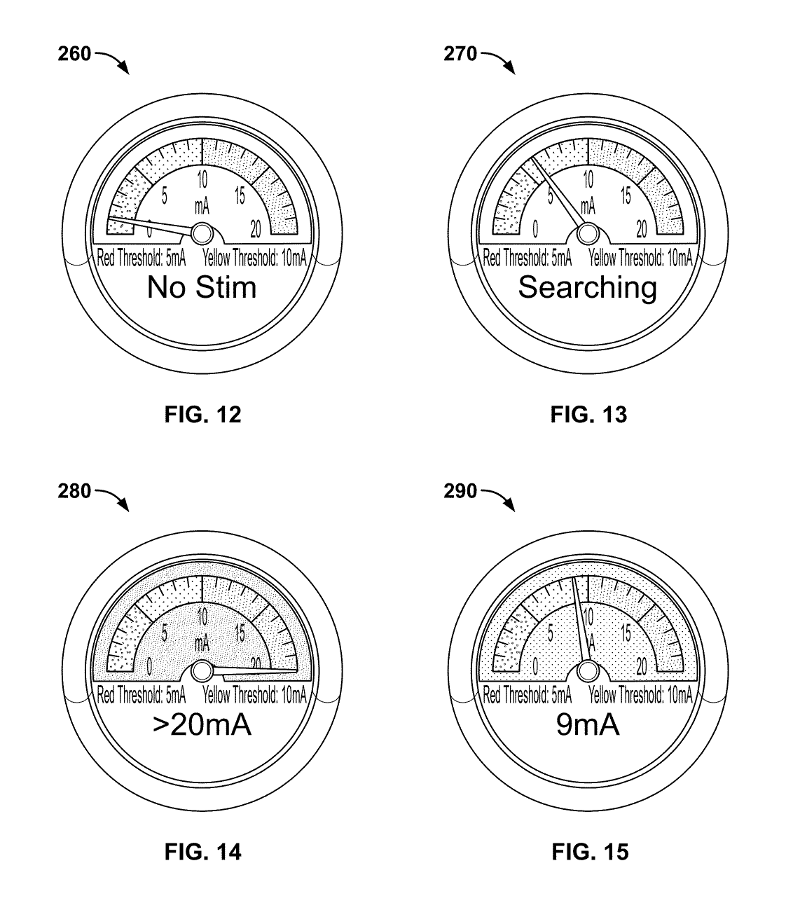

Various surgeon views 260, 270, 280, 290 are depicted in FIGS. 12-15 to illustrate exemplary changes to the dial that can occur during a surgical procedure. As shown in FIG. 12, when the selected surgical mode is running but the stimulus loop is not closed (e.g., the probe or other instrument is not touching the patient), the dial indicates “No Stim.” As shown in FIG. 13, when the stimulus loop is closed, but the algorithm has not yet identified a threshold, the dial indicates “Searching.” As shown in FIG. 14, when the algorithm reaches its maximum stimulus intensity without identifying a threshold, the dials indicates “>MAX,” where MAX is the maximum stimulus intensity for the mode, depicted as 20 mA in the figure. As shown in FIG. 15, when the algorithm has detected the minimum intensity required to produce a threshold crossing, that intensity (e.g., “9 mA” in the figure) is displayed and the background color of the dial may be adjusted as necessary.

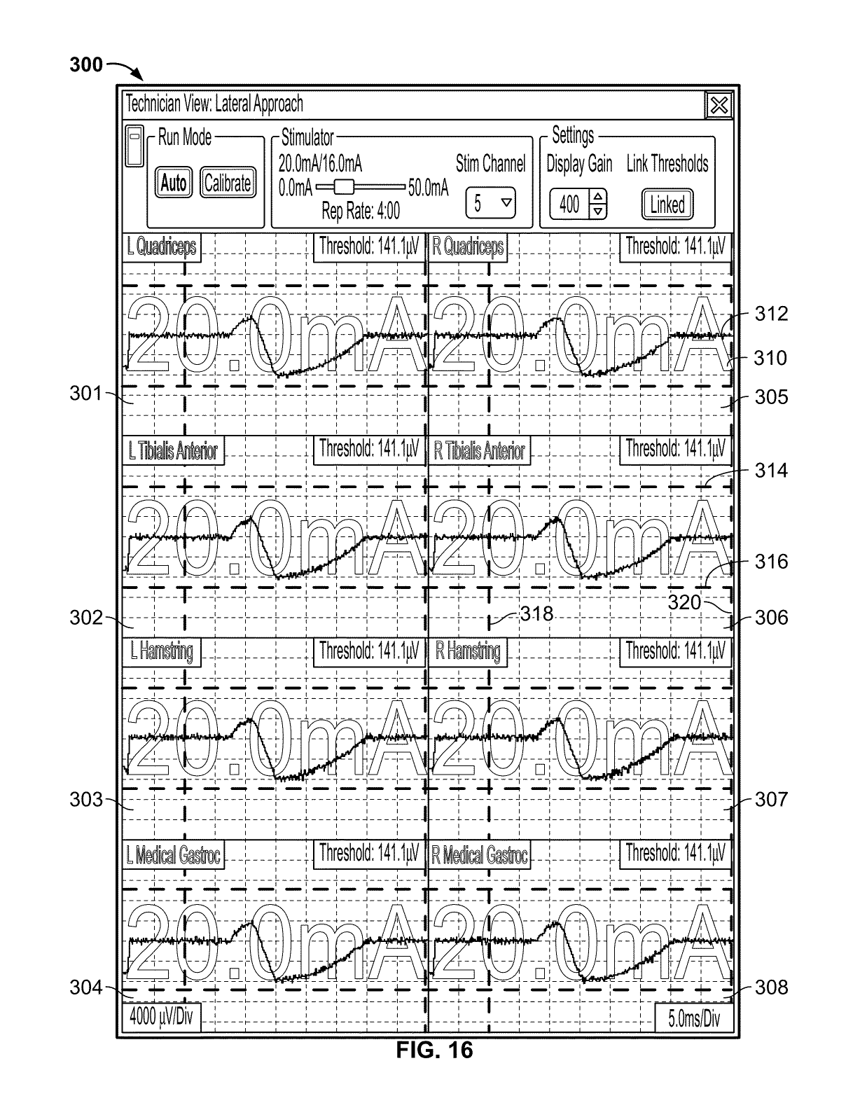

The monitorist view displays detailed information to the technician or monitorist, including the raw waveform responses for each sensed muscle. As discussed above, the monitorist view may include the waveform responses to the current stimulus while the surgeon view does not; instead including numeric and/or graphical indicators of distance and or current intensity based on the same waveform responses. The detailed information provided to the monitorist allows the monitorist to determine, for example, whether the information is reliable (e.g., by checking for artifacts or other signal noise) and adjust the settings of the monitoring system pre, post, or intraoperatively. As shown in FIG. 16, for example, the monitorist view 300 includes waveform responses and each sensed muscle has its own sub-window in the monitorist view. Eight sub-windows 301-308 are shown in the figure, one for each sensed muscle of the right and left leg, although any suitable number of sub-windows may be used. Responses within the monitorist view 300 are updated approximately once per second. The stimulus amplitude associated with each waveform is displayed via a colored watermark 310 in the background of the window 305 for that muscle. The waveforms displayed in each window (e.g., waveform 312 of window 305) may be determined based on a “threshold crossing” or a “response to last stimulus.” A threshold crossing occurs if the corresponding muscle evoked a suprathreshold response, and in such cases the response at the threshold value is displayed. A response to last stimulus occurs if the corresponding muscle did not evoke a suprathreshold response, and in such cases the response at the highest stimulus intensity is displayed. As an example, assume that the algorithm stimulated at 5, 6, 7, 8, and 9 mA during the one second period. The left quadriceps crossed the threshold at 6 mA, but none of the other muscles responded to any of the stimulus pulses. The left quad window would display its response at 6 mA, while the other muscle windows would display their responses at 9 mA. It is understood that displays are referred to as “monitorist views” or “surgeon views” in order to simplify the discussion and that any specific display may be viewed by a monitorist, surgeon, or other personnel associated with the surgical procedure.

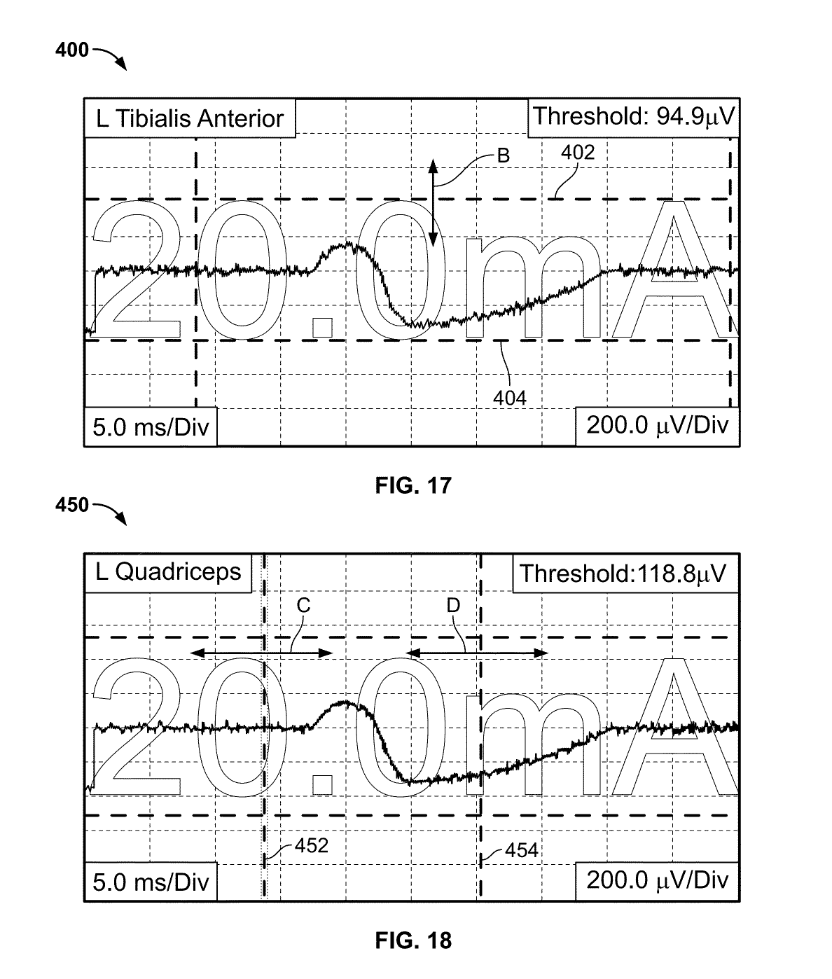

Within the windows 301-308 for each muscle, there is a pair of horizontal dashed lines (e.g., lines 314 and 316 of window 306) that represent the response amplitude threshold for that muscle. Responses that cross this dashed line in either the positive or negative direction will be counted by the algorithm as threshold responses. In certain embodiments, each channel has an independent response amplitude threshold. The response amplitude threshold can be adjusted by selecting one of the horizontal dashed lines and moving it up or down. The new response amplitude threshold level is indicated by the decorator in the top-right corner of that window. As shown in FIG. 17, the dashed lines 402, 404 of a given sub-window 400 can be moved up or down along the directions of arrow B.

Returning to FIG. 16, within the windows 301-308 for each muscle, there is also a pair of vertical dashed lines (e.g., lines 318 and 320 of window 306) that represent periods of time that are ignored by the algorithm. Specifically, any threshold crossings that occur before the left-most dashed line 318 are considered stimulus artifact and not a true muscle response. Any threshold crossings that occur after the right-most dashed line 320 are considered baseline drift artifact and not a true muscle response. As shown in FIG. 18, the dashed lines 452, 454 of a given sub-window 450 can be moved along the directions of arrows C and D.

In certain embodiments, threshold ranges are used to determine the colors displayed on the surgeon dial view and the audio tones that are played during the surgical procedure. These ranges can be adjusted by the monitorist or the surgeon. Any suitable threshold ranges may be used. For example, in certain embodiments where the maximum stimulus intensity is set at 20 mA, default threshold ranges of 0-5 mA, 5-10 mA, and greater than 10 mA may be used for color indications that are red, yellow, and green, respectively. Audio tones may accompany the procedure, and in certain embodiments a green threshold results in a single tone that repeats once every two seconds. For the yellow threshold, a single tone is produced at a relatively higher pitch, intensity, and repetition rate than the green tone. For the red threshold, a single tone is produced at a high pitch, intensity, and repetition rate than the yellow tone. It will be understood that any suitable color and/or audio scheme can be used to provide feedback to the surgeon during the surgical approach. As shown in FIG. 19, a threshold display screen 500 may be displayed that allows the user to change the threshold ranges for the red 502, yellow 504, and green 506 zones. In certain embodiments, the user can slide the respective threshold values up or down along the directions of arrow E. In certain embodiments, the user can change the threshold values by manually entering the desired threshold values (e.g., using the user interface 16 of FIG. 1).

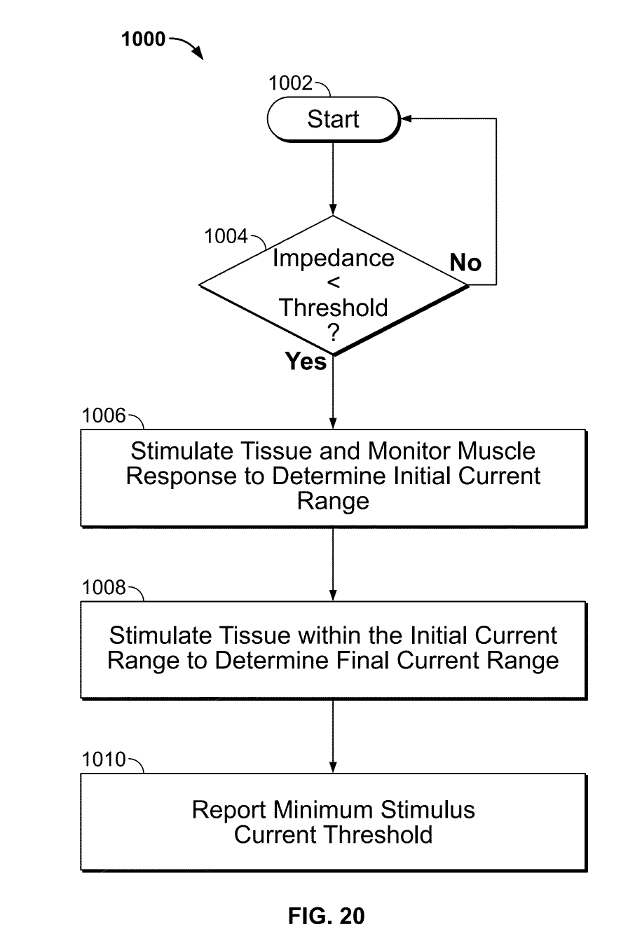

FIG. 20 shows a process flow 1000 for determining a minimum current that stimulates a nerve of interest and causes a neuromuscular response in a muscle that is innervated by the targeted nerve in accordance with certain implementations. Process 1000 may be employed during a surgical operation to determine one or more of a nerve proximity, nerve direction, nerve health pedicle integrity, or to perform another suitable assessment of the targeted nerve either simultaneously or in accordance with a user's selection of an operating mode. The illustrative process depicted in FIG. 20 employs an electrode disposed in the distal region of a probe or surgical tool to deliver a plurality of stimulus pulses at varying amplitudes and monitors responses (e.g., EMG signals) in one or more muscles innervated by particular nerves depolarized by the stimulus to determine the lowest amplitude current that provokes a neuromuscular response from one or more of the muscles. This threshold current can then be used to make the desired nerve assessment. Although the examples herein may focus on a single stimulus probe, the stimulus signals may be emitted from separate probes and the responses multiplexed to assess the nerves. Any suitable probe or surgical tool may be used, including without limitation, an electrified cannula through which other tools may introduced into the patient. Process 1000 may be employed while the probe or surgical tool is being introduced into a surgical site toward a nerve, or after the tool or probe has been positioned adjacent to the nerve. Process 1000 may also be used during assessment of pedicle screw procedures where the stimulus current is applied directly to a bone or to a screw that is already inserted or is yet to be inserted into the patient.

The process 1000 begins at step 1002 and monitors an impedance at step 1004 measured at an electrode used to deliver the stimulus pulses. In certain implementations, the impedance is monitored in a continuous loop, and no stimulus pulses are delivered from the electrode until the measured impedance drops below a predetermined threshold. The threshold-based impedance monitoring is a safety measure used to ensure that the electrode is positioned within a target tissue, rather than outside the tissue or in ambient air, before beginning to deliver electrical current to the electrode. When the electrode is in ambient air, a high impedance is detected, and the monitoring loop between steps 1002 and 1004 prevents the electrode from delivering electrical current. When the electrode is advanced into a surgical site or within a tissue, the measured impedance drops to a low level relative to the ambient impedance as contact with the tissue creates a continuous electrical path from the electrode. The drop to a low impedance level is detected by the threshold check at step 1004, and the process proceeds to step 1006 to begin the stimulus delivery stage of the process 1000. In an exemplary implementation, process 1000 includes a user-selectable input (such as a push button or switch) that may be selected or depressed by the user to indicate whether to begin delivering stimulus pulses. The switch or push button may be used in place of or in addition to the impedance-detection step 1004.

The stimulation delivery stage of process 1000 includes at least two parts, a first part during which an initial range around the target threshold stimulus current is determined and a second part during which the initial range is narrowed to a final range that includes the threshold current and has a size that is less than or equal to a predetermined resolution. As an illustrative example, in certain implementations the initial range may be fixed at 4 mA (that is, subsequent pulses applied after the starting point during the initial phase will be increased by fixed increments of 4 mA), and the final range resolution may be 1 mA current range. However, other ranges may be used for the initial range, for example ranges of less than 4 mA, 8 mA, 10 mA, or other suitable ranges, and other final resolutions may be used, for example 0.5 mA or less, less than 1 mA, 2 mA, greater than 2 mA, or other suitable resolutions, to suit specific applications of the process 1000.

At step 1006, in response to detecting a sufficiently low impedance, the nerve detection module causes a stimulus current is emitted from the electrode and into tissue in which the electrode is positioned. After each stimulus current is emitted, a muscle that is innervated by a nerve in or near the tissue is monitored, for example by EMG sensors or other sensors or detection systems, to determine whether or not the delivered stimulus evokes a response from the muscle. This determination is made by comparing the amplitude of the measured response, a voltage in the case of EMG monitoring, to a response threshold. If the measured response exceeds the threshold, then the stimulus that preceded the measured response is determined to be sufficient to stimulate the targeted nerve. The stimulus current starts at a low level and is increased for each subsequent stimulus until a measured response exceeds the response threshold. The stimulus current at which the measured response exceeds the response threshold is then used to define an initial current range which contains the minimum stimulus current that evokes a sufficient response from the monitored muscle. This initial current range is bound on the upper end by the stimulus current at which the response exceeds the threshold and on the lower end by the stimulus current that preceded the upper bound current. Because the response measured from the lower bound stimulus current did not exceed the threshold and the response measured from the upper bound stimulus current did exceed the threshold, the minimum current that will evoke a response that exceeds the threshold lies in this range.

Once the initial current range is determined, process 1000 moves to step 1008, in which subsequent stimulus currents and muscle measurements are used to narrow the initial current range into a smaller range that contains the minimum stimulus threshold. The stimulus currents delivered at step 1008 are selected from the initial current range, and a muscle response to each stimulus is measured to determine a final current range within the initial current range and having a lower bound at which a measured response does not exceed the response threshold and an upper bound at which a measured response does exceed the response threshold. The stimulus delivery and muscle response measurements are continued until the final range is narrowed to a size that is less than or equal to a predetermined resolution window, for example less than or equal to a 1 mA window or a 0.25 mA window, or any other suitable window.

After the final current range is determined, the upper bound of the final current range is reported as the minimum stimulus current at step 1010 to complete process 1000. In other implementations, another value within the final current range, for example the midpoint between the final upper and lower bounds, may be used as the reported minimum current. The reported current may then be presented to a medical specialist via a graphic interface or may be passed to another algorithm module for further processing to assess the target nerve, for example to estimate one or more of a nerve proximity, nerve direction, pedicle integrity, or to perform another suitable assessment of the targeted nerve.

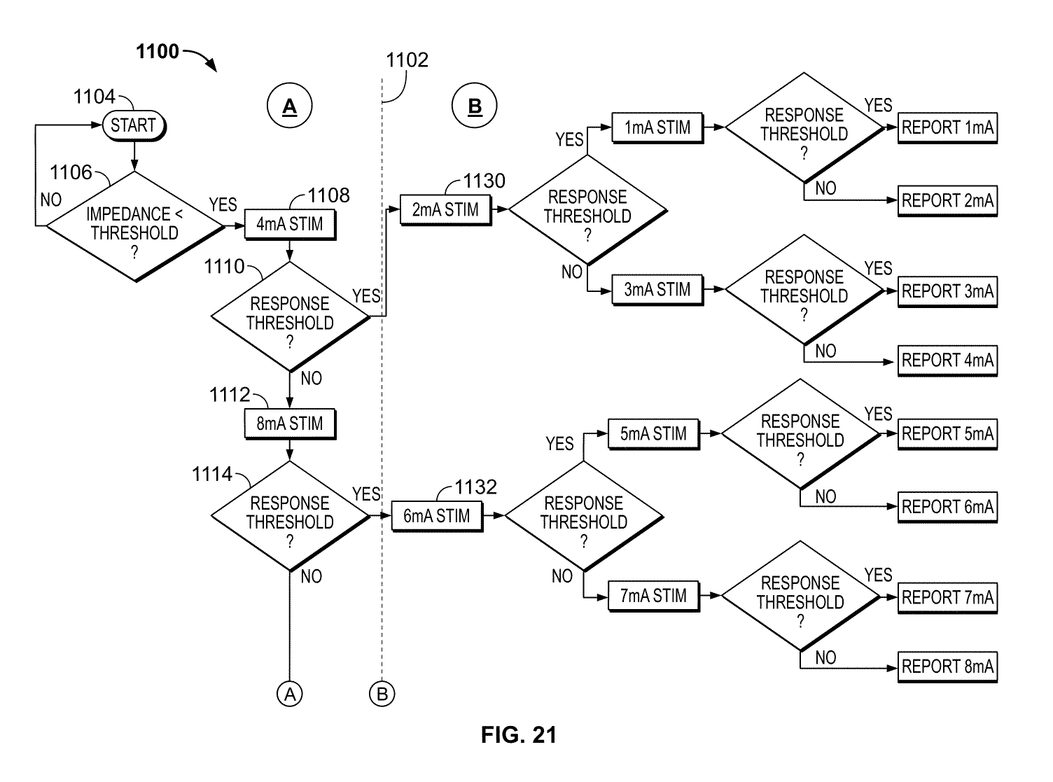

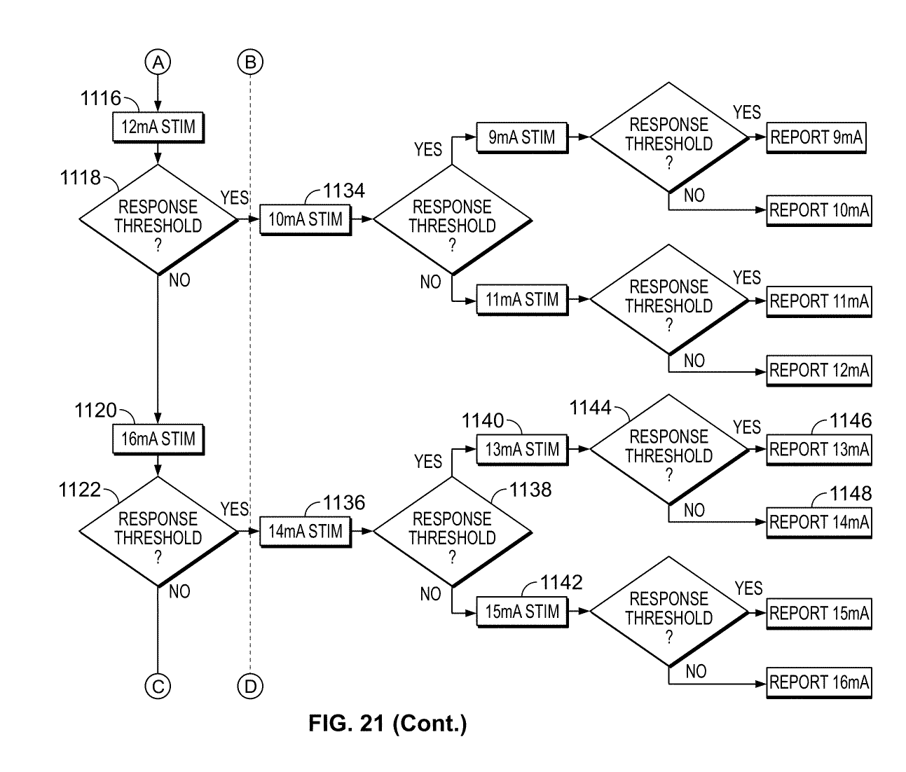

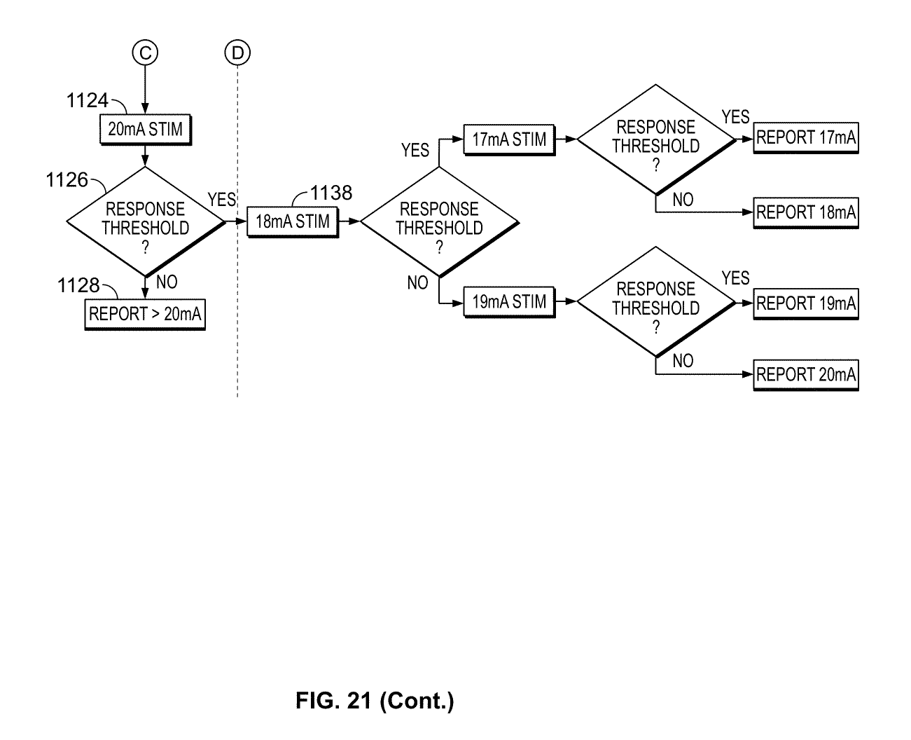

The currents delivered to the tissue in process 1000, and the increments or decrements between successive stimulus currents, can be adjusted and set to suit a particular application of the method or to improve threshold detection. An illustrative implementation of the process 1000 is shown in FIG. 21. The process 1100 in FIG. 21 is divided into two parts. During a first part A on the left of line 1102 in FIG. 21, stimulus current is delivered to the tissue and muscle responses are measured to determine an initial range containing the minimum threshold stimulus current, similar to step 1006 in process 1000 in FIG. 20. The current amplitude is increased by 4 mA for each successive stimulation during part A of the process up to a maximum stimulus current of 20 mA in order to efficiently determine the initial current range. At each successive stimulation, a muscle response is compared to a response threshold, and if a certain stimulus provokes a sufficient response then the initial range is defined between that last provoking stimulus and the previous delivered stimulus.

As shown in FIG. 21, part A of process 1100 begins at step 1104, and the impedance measured at a stimulus electrode is measured at step 1106. Similar to steps 1002 and 1004 of process 1000, a continuous loop between steps 1104 and 1106 forms a safety mechanism that reduces the chance of delivering electrical stimulation outside of a target tissue. When the electrode contacts tissue or the skin of a human or animal, the measured impedance drops below the threshold, and process 1100 advances to step 1108. At step 1108, a first stimulus having a predetermined amplitude is delivered from the electrode. The amplitude may be determined based on a prior threshold detected during a previous run of the algorithm. For example, since the minimum stimulation threshold of a nerve is not ordinarily expected to deviate significantly from a prior determination during a surgical procedure, the algorithm can begin a subsequent run by stimulating initially at the prior minimum current threshold determined for the nerve. If no such threshold has been determined, the process can begin by setting the first stimulation current at a predetermined current amplitude, e.g., 4 mA. In this case, the predetermined amplitude may be set by the processor running the algorithm (e.g., based on empirical information about the particular nerve to be targeted) or may be entered by the user. After the first stimulus current is applied, a muscle response is measured and compared to a threshold at step 1110. If the initial stimulus evokes a sufficient muscle response, the process 1100 moves to part B, discussed below, on the right side of line 1102. If the first stimulus does not evoke a sufficient response, the process 1100 moves to step 1112 for subsequent stimulus delivery.

At step 1112, the stimulus current is increased by a constant increment, and a second stimulus is delivered from the electrode at that current. At this step, the lower bound of the current range may be updated to the prior current amplitude applied at step 1108 above and the lower bound of the initial range may be updated to the amplitude of the second stimulus current. Thus, for example, if the current amplitude is increased by 4 mA at step 1112 when the initial current amplitude was 4 mA, then the new lower bound of the range may be updated to 4 mA and the new upper bound may be updated to 8 mA. Alternatively, only the upper bound may be tracked since the lower bound for the initial range can be easily determined by subtracting the value of the constant increment from the first stimulation current that evokes a response that exceeds the response threshold. A muscle response is measured and compared to a threshold at step 1114, and the process 1100 advances to part B if the response is sufficient. If the response is still below the maximum allowed threshold, then the stimulus current is again increased by the constant increment (e.g., by a 4 mA step to 12 mA), and a third stimulus current is delivered at that current in step 1116. A muscle response is measured and compared to the threshold at step 1118, and the process 1100 advances to either part B or to the next stimulus based on the comparison. The stimulus and response comparison cycle continues in part A through a 16 mA stimulus at step 1120, threshold comparison at step 1122, and a 20 mA stimulus at step 1124. If no muscle response exceeds the response threshold at any of the stimulus step, the process 1100 moves to step 1128 and reports a minimum threshold stimulus value of greater than 20 mA. At that point, it is determined that the minimum current required to evoke a sufficient muscle response is greater than the maximum allowed stimulus current (e.g., 20 mA), and the process 1100 ends.

When a muscle response exceeds the response threshold during part A of process 1100, the process moves to part B in which smaller current increases and decreases are used to identify the minimum threshold current, similar to step 1008 in process 1000. The stimulus current that evokes the sufficient response in part A is used to define the initial current range between that current and the previous delivered current, and the initial range determines the stimuli that are delivered to the tissue in part B. In process 1100, detection of a sufficient response at one of steps 1110, 1114, 1118, 1122, or 1126 define a 4 mA initial current range, and the stimuli delivered in part B are selected from within that 4 mA range. For example, if no sufficient stimulus is detected at steps 1110, 1114, or 1118 and a sufficient response is detected at step 1122, the 4 mA initial range is defined by the upper bound at which the sufficient response was detected (16 mA) and the lower bound at the previous delivered stimulus at which a sufficient response was not detected (12 mA). The stimuli in part B are then delivered at currents within the 12 mA-16 mA range until a narrower final range is determined.

In process 1100, the first stimulus delivered in part B is at an interior point within the initial current range, e.g., the midpoint, or a point closer to one of the boundaries of the initial current range. Each of the first stimuli in steps 1130, 1132, 1134, 1136, and 1138 thus bisect the initial current range and create two smaller ranges within the initial range, one of which contains the desired minimum stimulation current. For example, when a sufficient response is detected at step 1122 and the initial window of 12 mA-16 mA is defined, the first stimulus of part B is delivered at 14 mA in step 1136. This creates two new ranges, 12 mA-14 mA and 14 mA-16 mA, that may contain the minimum stimulus current, and a muscle response is measured and compared to the threshold at step 1138 to determine which of the two ranges is used for a next stimulus. The selected range is then again bisected. For example, if no threshold response is detected at step 1138, then the minimum current is above 14 mA, and the process 1100 proceeds to bisect the 14 mA-16 mA range by delivering a 15 mA stimulus at step 1142. If a sufficient response is detected at step 1138, then the minimum current is less than or equal to 14 mA, and the process 1100 proceeds to bisect the 12 mA-14 mA range by delivering a 13 mA current at step 1140. This process continues until a final range that has a width less than or equal to a set resolution is determined, and that final range is used to report the minimum current.

In process 1100, the resolution is 1 mA, and the 12 mA-14 mA range is bisected until a 1 mA range is determined. After the 13 mA stimulus at step 1140, muscle response is compared to a threshold at step 1144. If no sufficient response is detected, then the minimum current is within the range 13 mA-14 mA, and that range is the final current range having a width equal to the set resolution of 1 mA. If a sufficient response is detected at step 1144, then the minimum current is within the range 12 mA-13 mA. The process 1100 then reports a minimum current, using the upper bound of the final current range to report 13 mA at step 1146 or 14 mA at step 1148. In other implementations, a different value within the final range, for example the midpoint or another suitable value within the range, may be used for the reporting step.

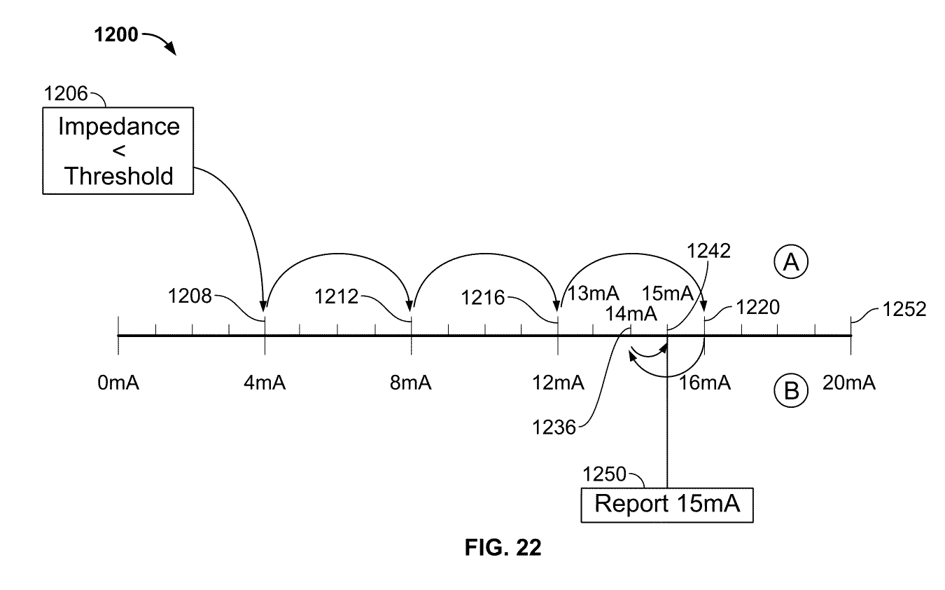

An example implementation of process 1100 is shown in the process 1200 of FIG. 22. The process 1200 begins by monitoring impedance measured at an electrode until the impedance drops below a safety threshold at 1206, similar to step 1106 of process 1100. The process 1200 then enters part A above the current scale 1252 with delivery of a first 4 mA stimulus 1208. A muscle response is measured, and when no sufficient response is detected the stimulus is incremented by 4 mA to deliver the second stimulus 1212 at 8 mA. Again no sufficient muscle response is detected, and the stimulus is again increased by 4 mA to deliver another stimulus 1216 at 12 mA. After the 12 mA stimulus, no sufficient muscle response is detected, and another stimulus 1220 is delivered at 16 mA. After this stimulus, a muscle response exceeding the response threshold is detected, and the initial current range is defined from 12 mA (no sufficient response detected) to 16 mA (sufficient response detected). The process 1200 then moves from part A to part B below the scale 1252 and begins to bisect the initial current range until a final current range equal to the resolution of 1 mA is determined.

Part B of process 1200 starts with a 14 mA stimulus 1236, halfway between the 12 mA lower bound and the 16 mA upper bound of the initial current range. After the 14 mA stimulus 1236, no sufficient muscle response is detected, and the minimum threshold stimulus lies within the 14 mA-16 mA range. This range is bisected with a next stimulus 1242 delivered at 15 mA, and the muscle response measured after stimulus 1242 is used to determine which range, 14 mA-15 mA or 15 mA-16 mA, defines the final current range with 1 mA resolution. In the process 1200, a sufficient muscle response is detected, and 15 mA is reported as the minimum stimulus threshold at 1250.

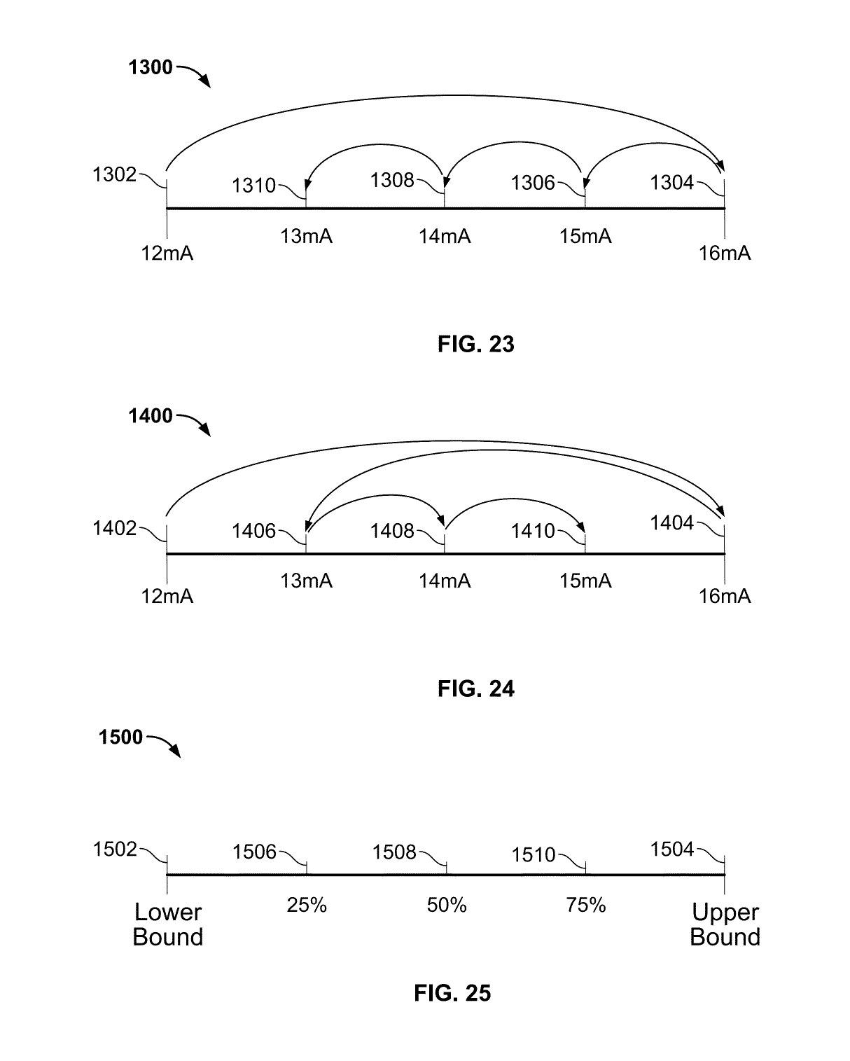

In addition or as an alternative to the bisecting resolution, other resolution approaches may be employed once an initial current range is determined. One alternative resolution phase approach 1300 is shown in FIG. 23. In this approach, an initial current range of 12 mA-16 mA is determined after no sufficient muscle response is detected for a 12 mA stimulus 1302 and a sufficient muscle response is detected for a 16 mA stimulus 1304. After the 16 mA stimulus, the resolution phase begins by decrementing the stimulus by 1 mA to a 15 mA stimulus 1306. If a sufficient muscle response is detected at the 15 mA stimulus, the stimulus is again decremented to a 14 mA stimulus 1308. If no sufficient muscle response is detected at the 15 mA stimulus, then a final current range of 15 mA-16 mA is determined, and 16 mA is reported as the minimum stimulus current. The decrementing continues until either no response is detected at 14 mA stimulus 1308, or the 13 mA stimulus 1310 is reached. If the 13 mA stimulus 1310 is delivered, a sufficient muscle response results in a report of 13 mA as the minimum stimulus current, while no sufficient muscle response results in a report of 14 mA. In addition to the 1 mA decrements shown in FIG. 23, other decrement values may be used in similar resolution approaches. For example, the decrement amount may be set equal to the resolution range, and adjustment of the resolution may likewise change the decrement amount employed during this phase.

While the resolution approach shown in FIG. 23 begins near the upper bound of the initial range and decrements for each subsequent resolution stimulus, it may be preferred to instead begin near the lower bound of the range and increment the stimulus delivered each time. Such an approach may be employed in the interest of patient safety, for example, to avoid delivering higher currents near the upper bound in case the threshold current is near the lower bound and a suitable final current range can be determined using only lower stimuli.

FIG. 24 shows a resolution approach 1400 that increments from the lower bound of an initial range rather than decrementing from an upper bound of the range. After no sufficient muscle response is detected at 12 mA stimulus 1402 and a sufficient muscle response is detected at 16 mA stimulus 1404, an initial window of 12 mA-16 mA is defined for resolution. In approach 1400, the resolution begins by returning to 12 mA stimulus 1402 and incrementing by a set value, 1 mA in approach 1400, to deliver the first resolution stimulus 1406 at 13 mA. If a sufficient muscle response is detected after this stimulus, a final range of 12 mA-13 mA is determined and the process ends without delivering higher stimulus currents. If no sufficient muscle response is detected, then the minimum threshold current is greater than 13 mA, and the approach 1400 increments to a 14 mA stimulus 1408 and, if needed, a 15 mA stimulus 1410 to determine the final range and report a minimum threshold current.

In addition to constant increase and decrease and bisecting approaches, other approaches may be employed to suit particular applications. In some implementations, the increment or decrement steps applied to obtain the subsequent stimulus amplitude are determined based on the magnitude of the immediately preceding stimulus amplitude. In such implementations, if the preceding stimulus amplitude is greater than or equal to a first threshold (e.g., 24 mA), the subsequent amplitude is increased or decreased by a larger fixed step (e.g., 8 mA rather than 4 mA). Similarly, if the preceding stimulus amplitude is less than a first threshold (e.g., 8 mA), the subsequent amplitude is increased or decreased by a smaller fixed step (e.g., 2 mA rather than 4 mA). For example, it may be desirable to provide a resolution approach that is responsive to the muscle responses detected during identification of the first current range or to the values determined for the upper and lower bounds of the first current range. FIG. 25 shows one example of these responsive approaches. In approach 1500, an initial current range is determined between a lower bound 1502 at which no sufficient muscle response is detected and an upper bound 1504 at which a sufficient muscle response is detected. The range is divided up into quarters, with potential first resolution stimulus currents 1506, 1508, and 1510 at 25%, 50%, and 75%, respectively, of the initial range. The approach 1500 selects on of the three stimuli 1506, 1508, and 1510 to use for the first resolution stimulus. For example, if the muscle response detected when stimulus is delivered at upper bound 1504 only slightly exceeds the response threshold, that response may indicate that the minimum stimulus current is nearer to the upper bound 1504 than it is to the lower bound 1502. In that situation, the approach 1500 may select the 75% stimulus 1510 to use for the first resolution stimulus to efficiently locate the minimum current. On the other hand, if the response at upper bound 1504 exceeds the response threshold by a large margin, that response can indicate that the minimum stimulus is nearer to the lower bound 1502, and the system may choose the 25% stimulus 1506 to efficiently find the minimum current.

The selection between the three stimulus currents 1506, 1508, and 1510 may be aided by the use of secondary thresholds used in addition to the response threshold employed to determine the initial current range. For example, three secondary thresholds may be defined, with each threshold being greater than the response threshold and corresponding to one of the stimulus currents 1506, 1508, and 1510. When the muscle response at upper bound 1504 is detected, it may also be compared to the secondary thresholds, and the stimulus current corresponding to the highest of the secondary thresholds that is exceeded may be chosen for the first resolution stimulus.

In the nerve assessment process, such as the process 1000 shown in FIG. 20, different stimulus current adjustment approaches may also be employed to determine initial current ranges before resolution begins. Such approaches may balance a trade off between efficiently defining the initial range and maintaining safety for a patient. This balance can be managed by using different size stimulus increases for lower stimulus currents and higher stimulus currents. A large jump at low currents, for example from 0 mA to 16 mA, my harm a patient, particularly when the minimum stimulus current is low, for example 2 mA. The same jump, however, may not harm a patient at higher current, for example jumping from 32 mA to 48 mA when no sufficient muscle response is detected at 32 mA. Thus, it may be preferable to use a smaller current increase at the lower currents and switch to larger increases at the higher currents to maintain both safety and efficiency.

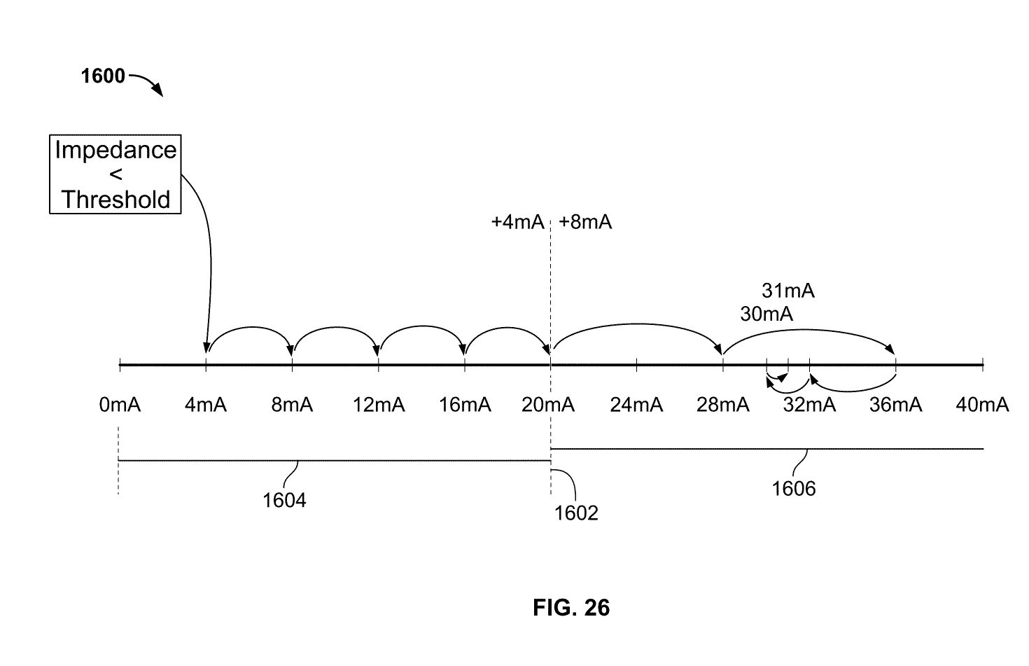

FIG. 26 shows an alternate approach 1600 for defining an initial current range during nerve assessment. In this approach, a maximum stimulus current is set to be greater than 40 mA, and thus constant steps of a small size may not provide an efficient determination of the initial range. As shown in the approach 1600, two different increase sizes are employed, with 4 mA increases used below 20 mA stimulus 1602 and 8 mA increases used above 20 mA stimulus 1602. This approach may be beneficial if, for example, the targeted nerve may suffer damage from a jump from 0 mA to 8 mA but will not suffer damage from a jump from 0 mA to 4 mA or a jump from 20 mA to 28 mA. In the approach 1600, the 4 mA increase is used during part 1604 up until the 20 mA stimulus 1602, similar to the increases in part A of FIG. 20 discussed above. If no sufficient muscle response is detected at the 20 mA stimulus, the approach 1600 then switches to an 8 mA increase in stimulus current for part 1606 above 20 mA and continues to increase at 8 mA until a sufficient muscle response is detected, for example at 36 mA. The detected response then defines an 8 mA initial range, and any suitable resolution approach may be used to narrow the 8 mA range down to a final range within the predetermined resolution settings.

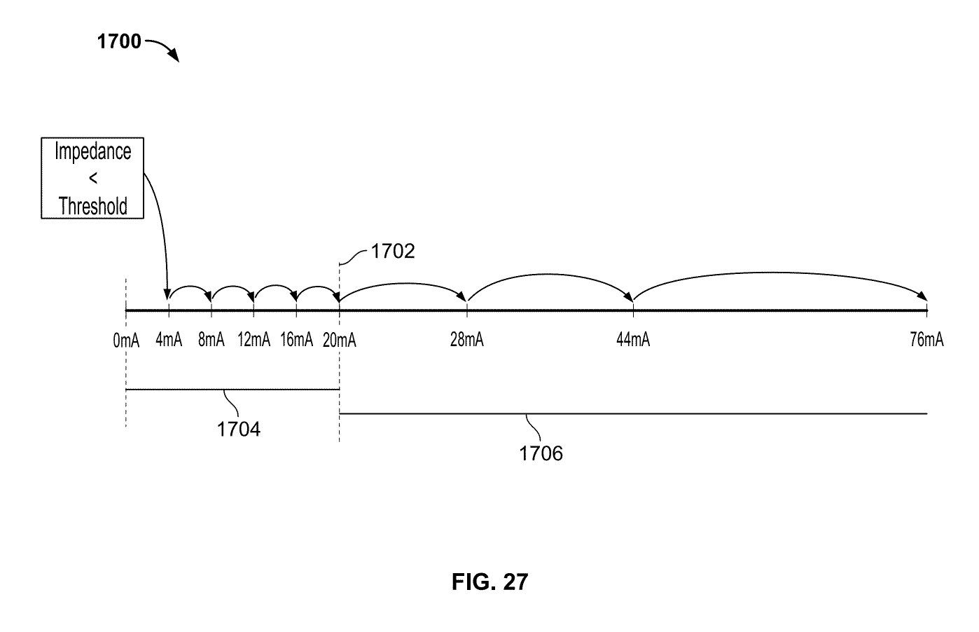

In addition to the approach 1600 shown in FIG. 26, larger stimulus increases may be used for each stimulus above a certain current to more quickly find the initial current range. FIG. 27 shows an approach 1700 in which 4 mA increases are used for each stimulus delivered during part 1704 up to 20 mA stimulus 1702, after which larger increases are used in part 1706. After no sufficient muscle response is detected at 20 mA stimulus 1702, the stimulus increase size doubles for each subsequent stimulus. For example, the stimulus increases by 8 mA from 20 mA to 28 mA, then by 16 mA from 28 mA to 44 mA, then by 32 mA from 44 mA to 76 mA. This may continue until either a maximum stimulus current is reached or a sufficient muscle response is detected following a stimulation. When the sufficient muscle response is detected, an initial window is defined and has a size equal to the size of the last increase used in the approach. Any suitable resolution approach may then be employed to narrow that range down to the final current range.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in spinal surgical procedures, may be applied to systems, devices, and methods to be used in other surgical procedures performed in the proximity of neural structures where nerve avoidance, detection, or mapping is desired, including, but not limited to selected brain surgeries, carotid endarterectomy, otolaryngology procedures such as acoustic neuroma resection, parotidectomy, nerve surgery, or any other surgical procedures.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. A surgical monitoring system comprising:
a first display device;
a second display device; and
a base unit in communication with the first display and the second display and having a processor configured to:
receive a nerve detection signal that includes components of a neuromuscular response detected by a sensor disposed near a muscle innervated by a nerve being monitored;
extract from the nerve detection signal a first component of the neuromuscular response corresponding to a first time period and a second component of the neuromuscular response corresponding to the first time period, wherein the first and second components are different; and
transmit the first component to the first display device and the second component to the second display device for substantially simultaneous display during a surgical procedure.

2. The surgical monitoring system of claim 1, wherein the first component comprises a waveform corresponding to the neuromuscular response and the second component does not include the waveform.

3. The surgical monitoring system of claim 2, wherein the second component comprises a numerical value of a current intensity level that elicited the neuromuscular response and the first component does not include the numerical value of the current intensity that elicited the neuromuscular response.

4. The surgical monitoring system of claim 1, wherein the first and second display devices display information using different respective graphical interfaces.

5. The surgical monitoring system of claim 4, wherein the second display device displays at least some, but not all, information that is simultaneously displayed by the first display device.

6. The surgical monitoring system of claim 5, wherein the first display has a number of windows, and the second display has fewer windows than the first display.

7. The surgical monitoring system of claim 4, wherein the first display has selectable items that configure the graphical interface in which information is displayed in the first display.

8. The surgical monitoring system of claim 1, wherein the first display device displays waveform responses for a plurality of muscles.

9. The surgical monitoring system of claim 8, wherein the first display device displays at least one of the approximate distance to and direction of the nearest nerve.

10. The surgical monitoring system of claim 8, wherein the second display device displays a virtual representation of at least one nerve and a direction from the probe to the nerve.

11. The surgical monitoring system of claim 8, wherein the second display device indicates the lowest threshold current for each of the plurality of muscles at a given point in time.

12. A method for monitoring a nerve during a surgical procedure, comprising:
receiving a nerve detection signal that includes components of a neuromuscular response detected by a sensor disposed near a muscle innervated by a nerve being monitored;
extracting from the nerve detection signal a first component of the neuromuscular response corresponding to a first time period and a second component of the neuromuscular response corresponding to the first time period, wherein the first and second components are different; and
causing a first display device and a second display device to display the first component and the second component, respectively, in a substantially simultaneous fashion during a surgical procedure.

13. The device of claim 7, wherein the processor is configured to:
receive an indication of a user input for one of the selectable items in the first display; and
adjust the first component of the neuromuscular response that is extracted from the nerve detection signal and transmitted to the first display device based on the user input.

14. The device of claim 13, wherein the processor is configured to adjust the second component of the neuromuscular response that is extracted from the nerve detection signal and transmitted to the second display based on the user input.

15. The device of claim 13, wherein the user input comprises a selection of a particular muscle or nerve, and the processor is configured to extract neuromuscular response information for the particular muscle or nerve from the nerve detection signal in response to receiving the user input.

16. The device of claim 15, wherein the process is configured to transmit an indication of the neuromuscular response of the particular muscle or nerve to the second display device.

17. The device of claim 16, wherein the indication transmitted to the second display device comprises at least one of an EMG waveform, a numerical current value, an indication of a current range, or an indication of a current threshold.

18. The device of claim 1, wherein the first component extracted from the nerve detection signal comprises neuromuscular response data for a first set of muscles, and the second component extracted from the nerve detection signal comprises neuromuscular response data for a second set of muscles.

19. The device of claim 18, wherein the second set of muscles includes at least one but fewer than all of the muscles of the first set of muscles.

20. The device of claim 1, further comprising a central base unit configured to adjust both of the first and second components extracted from the nerve detection signal based on user input.
